(19) United States
(12) Patent Application Publication
(10) Pub. No.: US 2020/0216515 A1
(43) Pub. Date: Jul. 9, 2020

(54) METHODS OF ANTIGEN-DEPENDENT CHIMERIC ANTIGEN RECEPTOR (CAR) IMMUNE CELL SELECTION

(71) Applicant: CELLECTIS, Paris (FR)

(72) Inventors: Philippe DUCHATEAU, Draveil (FR); Anne-Sophie GAUTRON, Etrechy (FR); Laurent POIROT, Paris (FR)

(21) Appl. No.: 16/633,604

(22) PCT Filed: Jul. 26, 2018
(86) PCT No.: PCT/EP2018/070258

§ 371 Date: Jan. 24, 2020

Related U.S. Application Data
(60) Provisional application No. 62/537,435, filed on Jul. 26, 2017.

(30) Foreign Application Priority Data
Sep. 22, 2017 (DK) PA201770718

(51) Int. Cl. C07K 14/725; C12N 15/10

(52) U.S. Cl. CPC C07K 14/7051; C12N 15/1037; A61K 38/00; C12N 2510/00; C07K 2317/24; C07K 2319/03

(57) ABSTRACT

The present disclosure provides in vitro and in vivo methods for selecting a candidate CAR polynucleotide to be expressed in immune cells for its preferential capability to make immune cells proliferate in an antigen-dependent manner.

### FIELD OF THE INVENTION

The field of the invention relates generally to the field of immunology and cancer. In particular, the field of the invention relates to methods of screening chimeric antigen receptor (CAR) immune cells.

### BACKGROUND

Adoptive cell therapy using T-cells engineered to express chimeric antigen receptors (CARs) that target tumor cells bearing antigens recognized by the CARs is a promising new treatment strategy for cancer. However, one of the problems is that standard short term (e.g., 4 hours) in vitro cytotoxicity assays to assess antigen dependent CAR T cell function do not always correlate with results shown in vivo. Different CAR architectures can exhibit similar activity in vitro while performing quite differently in vivo, which cannot be predicted. Therefore, it is usually necessary to perform costly and time consuming in vivo assays to screen or compare activity of multiple CAR constructs.

Thus, there is a significant need for new and sensitive assays that measure CAR immune cell activity and which enable selection of CAR polynucleotides to be expressed in immune cells for their preferential capability to make immune cells proliferate in an antigen-dependent manner.

This background information is provided for informational purposes only. No admission is necessarily intended, nor should it be construed, that any of the preceding information constitutes prior art against the present invention.

### SUMMARY

It is to be understood that both the foregoing general description of the embodiments and the following detailed description are exemplary, and thus do not restrict the scope of the embodiments.

In one aspect, the invention provides an in vitro method for selecting a candidate CAR polynucleotide to be expressed in immune cells for its preferential capability to make immune cells proliferate in an antigen-dependent manner, comprising one or several of the following steps:
i) providing a population of immune cells endowed with a variety of CAR polynucleotides targeting the same antigen;
ii) incubating the population of CAR immune cells under i) with target cells expressing the same antigen for a period of time;
iii) adding an additional quantity of target cells to the incubated CAR immune cells and incubating for an additional period of time;
iv) optionally repeating step iii) one or more times;
v) detecting the presence of an enriched sub-population(s) of CAR immune cells by sequencing, preferably deep-sequencing, or amplifying the polynucleotides encoding the various types of CARs; and
vi) selecting the CAR polynucleotide expressed by said enriched sub-population of CAR immune cells.

In another aspect, the invention provides an in vivo method for selecting a candidate CAR polynucleotide to be expressed in immune cells for its preferential capability to make immune cells proliferate in an antigen-dependent manner, comprising
i) providing a population of immune cells endowed with a variety of CAR polynucleotides targeting the same antigen;
ii) administering the population of immune cells to a subject having target cells comprising the antigen, wherein a CAR immune cell sub-population that exhibits a preferential capability to proliferate in an antigen-dependent manner becomes enriched in the population of CAR immune cells;
iii) detecting the presence of the enriched sub-population of CAR immune cells; and
iv) selecting the CAR polynucleotide expressed by said enriched sub-population of CAR immune cells.

So far, classical CAR screening procedures have laid on individual experiments to test each CAR in-vitro functionalities, such as by flow cytometry based cytotoxicity and degranulation assays, IFN gamma secretion in the culture supernatants from separate batches. A large number of cells were required to produce reliable and comparable data in this way, which was critical in terms of sourcing the cells, especially when the experiments are run with primary cells. Indeed, a large variability has been observed between donors, so that the cells have to come from a single donor for the comparisons to be meaningful. According to the present invention, immune cells endowed with different CAR constructions are pooled together in-vitro or in-vivo. The proportion of the respective populations of CAR immune cells is monitored over time by sequencing analysis upon rounds of immune cells activation. By sequencing analysis is meant that the polynucleotide sequences coding for the CAR are qualitatively and/or qualitatively assessed to control their overall frequency. This is by contrast to the prior art methods where the potency of the CAR T-cells was assessed with respect to their CAR polypeptide expression. Deep-sequencing is a technique of choice but other techniques are known in the art, such as quantitative PCR.

Immune cells activation is preferably operated with irradiated cells which are added to the medium or injected to the animal at a predetermined ratio target cell/CAR positive cell. After the last round of activation, the cells are analyzed to determine whether specific CARs have led to a dominant population of immune cells. The methods of the present invention are particularly useful to screen genetically engineered immune cells to test their competitive advantage. To be more realistic, the method of the present invention can also be performed in-vivo in different animal models with tumors, in the presence of allogeneic immune cells, drugs, immune depletion treatments at various doses, to test different immune cell attributes of those engineered cells such as their resistance to drugs, alloreactivity, persistence, cytokine release, . . .

Among its plurality of advantages, the method of the present invention allows only one experiment set-up to rank CAR immune cells based on their activation/proliferation capabilities. CAR enrichment can be measured by deep sequencing after antigen specific proliferation. Only few cells are needed, in general less than 10⁷, more generally less than 4.10⁶, which can be sourced from one donor by leukapheresis. Less variability is observed between replicates because CAR immune cells are initially pooled in the same culture conditions (prevent variations linked to evaporation, pipetting errors, homogenization, experimental mistakes . . . ). Analysis is based on relative frequencies: there is no bias on cell counting.

The present method is particularly useful to compare antigen dependent activation/proliferation of CAR immune cells that have different genetic background. For instance, CAR immune cells that have been genetically engineered, sometimes deriving from stem cells (iPS or HSCs), can harbor genetic modifications (mutations or transgene integration), such as to repress or inactivate the expression of TCR, B2m or immune checkpoints (ex: PD1 or CTLA4) or other useful genes to improve their therapeutic potency.

FIGS. 21, 22, 23 and 24 illustrate and supplement the different embodiments and alternatives of the methods described herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1: 3 days after activation, T cells were transduced with the CD22 tool CAR (m971) and 4 different candidates at a MOI of 5. 14 days post transduction CAR expressions were assessed by flow cytometry on viable T cells using a recombinant protein targeting the whole extracellular domain of CD22. The frequency of positive cells is indicated in each panel.

FIG. 2: 4 days after reactivation, CAR expression among the mix of CD22 transduced-T cells were assessed by flow cytometry on viable T cells using a recombinant protein targeting the whole extracellular domain of CD22. The frequency of positive cells is indicated in each panel. The 3 quadrants for reactivation by NALM-16 (CD22 positive target cell line) and by SupT1 (CD22 negative cell line) correspond to triplicates.

FIG. 3: 3 days after the second reactivation, CAR expression among the mix of CD22 transduced-T cells were assessed by flow cytometry on viable T cells using a recombinant protein targeting the whole extracellular domain of CD22. The frequency of positive cells is indicated in each panel.

FIG. 4: 3 days after the third reactivation, CAR expression among the mix of CD22 transduced-T cells were assessed by flow cytometry on viable T cells using a recombinant protein targeting the whole extracellular domain of CD22. The frequency of positive cells is indicated in each panel.

FIG. 5: Alignment of the scFv sequences of 5 CARs of CD22. The differences between the 5 constructions appear in white or grey. The sequences of the primers chosen are displayed above the alignment (in bold, illumina adaptors; in light, oligo sequences).

FIG. 6: Summary of the results obtained by deep sequencing analysis. Each bar represents the relative frequency of each CAR among the mix of the 5 CARs at the different steps of reactivation (upper panel: +SupT1 negative target cells; bottom panel: +NALM-16 positive target cells). The relative frequency was calculated as per the following formula: Relative frequency=(# of analyzable reads for a given CAR/# of total analyzable reads)×100. Error bars correspond to triplicates of each time point.

FIG. 7: In vitro functional activities of the chosen CARs (cytotoxicity assay, frequencies of CAR candidates, degranulation assay and IFN-γ measurement). The results obtained in this first screen highlighted that the 3 best candidates were m971, A-D4 and B-B7 CARs.

FIG. 8: Serial killing assay results obtained with two different batches of CAR T cells (#1 and # 2) over 12 days pooled in-vivo.

FIG. 9.: In vivo antitumor activity of CAR T cells (#1 and #2) in BRGS mice.

FIGS. 10 and 11: Example of 2 CARs that showed similar profiles in a standard cytotoxicity assay that showed different behaviors in the serial killing assay in-vivo. As shown in FIG. 10, these CARs had similar cytolytic activity after 4 h of coculture with target cells (NALM16, CD22⁺). However, as shown in FIG. 11, these cells could be discriminated after the serial killing assay. #4 maintained cytolytic activity for a longer time than #3.

FIGS. 12 and 13: Detailed “QR3” (FIG. 12) and “R2” (FIG. 13) architectures of CARs targeting CLL1, which have been assayed according to the invention as reported in Example 2.4.

FIG. 14: Degranulation assay of different CARs with several cell lines as detailed in Example 2.3.

FIG. 15: IFN-γ release obtained upon activation with the different anti-CLL1 CARs after co-culture for 24 h with several cell lines as detailed in Example 2.3.

FIG. 16: Cytotoxicity assay. The cytolytic activity of CART cells as assessed after 4 h coculture with the different target cells (EOL, HL-60 and JEKO) and normalized to TA as detailed in Example 2.3.

FIG. 17: Kinetics of M2-QR3 and M26-QR3 CAR expression. CART cells were detected at different timings using Rituximab as detailed in Example 2.3.

FIG. 18: Degranulation assay obtained with M2-QR3 and M26-QR3 CARs. CAR T cells were co-cultured with target cells (EOL, HL-60, U937 and JEKO) for 6 hours and CD107a expression was checked on the surface of CAR CD8 T cells as detailed in Example 2.3.

FIG. 19: Cytotoxicity assay. The cytolytic activity of both candidates M2-QR3 and M26-QR3 CARs were tested with several cell lines at distinct ratios and different timepoints post transduction as detailed in Example 2.3.

FIG. 20: Expression of activation marker CD25 obtained with M2-QR3 and M26-QR3 CARs at different timepoints (day 11 and day 14) post transduction as detailed in Example 2.3.

FIG. 21: Schematic representation of the steps and time lines of one selection method according to the invention as pursued in Example 2.4. According to this embodiment, initial cell culture (ex: primary cells, such as PBMC from donors) are split into different subcultures, each undergoing (1) transfection with a rare-cutting endonuclease targeting a locus (ex: TRAC, PDCD1, B2M loci) for insertion of the CAR candidate sequences, and shortly after (2) transduction with viral vectors (ex: rAAV6 vector) comprising sequences encoding the different CAR candidates. After a period of separate cultures up to 18 days, CAR expression is measured at the surface of the immune cells. The different cultures are then pooled together based on the level of CAR expression, so that CAR candidates are equally represented in the culture. The immune cells are then activated by contact with target cells, which have been irradiated, at a selected ratio (ex: 1:1). The cell culture can be split into several replicates and regularly reactivated (ex: every 3 days) by adding irradiated cells at the selected ratio. After 20 to 35 days, preferably between 25 and 30 days, sequence analysis is performed to assess the proportion of CAR candidates dominating the culture. In Example 2.4, the competition is performed between four candidates M2-QR3, M26-QR3, M2-R2 and M26-R2 CARs.

FIG. 22: Schematic representation of a variation of the method according to the invention presented in FIG. 21, where the initial sub-cultures undergo a retroviral transduction respectively with vectors encoding different CAR candidates.

FIG. 23: Schematic representation of the steps and time lines of one method according to the invention, wherein the initial immune cells culture is transduced with a library of viral vectors comprising sequences encoding different CAR candidates. The different candidates in the library are in equimolar concentration, so that the transduced cells form a mixed population of CAR positive cells. They might not be equally represented in this population before activation if they don't grow the same way. One (or more) could take over the others. At this stage, a deep-sequencing analysis at the day you reactivate them may be performed. And actually, it is interesting to know if there is an enrichment just during the expansion phase for example. And then rounds of reactivation may be carried out as described.

FIG. 24: Schematic representation of a variation of the method illustrated in FIG. 23, where the transduction step is combined with a transfection step introducing a rare-cutting endonuclease for insertion of the CAR candidates at a preselected locus. According to a preferred embodiment of the invention this locus is TCRalpha and/or TCR beta to inactivate or reduce TCR expression, which makes T-cells less alloreactive as previously described in WO2013176915.

FIG. 25: Flow cytometry analysis to assess the proportion of CAR immune cells in the initial cultures prior to pooling as detailed in Example 2.4. 3 days after activation, individual sub-cultures of T cells were transduced with 4 different anti-CLL1 CAR candidates M2-QR3, M26-QR3, M2-R2 and M26-R2 respectively at a MOI of 30000 vg/cell. 14 days post transduction, CAR expressions were assessed by flow cytometry on viable T cells using a recombinant protein targeting CLL1. The frequency of positive cells is indicated in each panel.

FIG. 26: Flow cytometry analysis to assess the proportion of the CAR positive cells in the three replicate cultures (R1, R2 and R3). 4 days after reactivation with irradiated HL60 (positive targets) or Jeko (negative targets), CAR expression among among the mix of CLL1 transduced-T cells was assessed by flow cytometry on viable T cells using a recombinant protein targeting CLL1 as detailed in Example 2.4. The frequency of positive cells is indicated in each panel. As a negative control, the pool of CART cells is kept in culture without any kind of target cells.

FIG. 27: Flow cytometry analysis to assess the proportion of the CAR positive cells in the three replicate cultures (R1, R2 and R3) 3 days after the second reactivation with HL60 irradiated cells or JEKO (CLL1 negative cells) and T-cells (negative control) CAR expression among the mix of CLL1 transduced-T cells was assessed by flow cytometry on viable T cells using a recombinant protein targeting CLL1 as detailed in Example 2.4. The frequency of positive cells is indicated in each panel.

FIG. 28: Flow cytometry analysis to assess the proportion of the CAR positive cells in the three replicate cultures (R1, R2 and R3) 4 days after the third reactivation with HL60 irradiated cells . CAR expression among the mix of CLL1 transduced-T cells was assessed by flow cytometry on viable T cells using a recombinant protein targeting CLL1 as detailed in Example 2.4. The frequency of positive cells is indicated in each panel.

FIG. 29: Alignment of the scFv sequences of the 4 CARs of interest. The differences between the 4 constructions appear in white or grey. The sequences of the primers chosen are displayed above the alignment (in bold, illumina adaptors; in light, oligo sequences)

FIG. 30 : Summary of the results obtained by deep sequencing analysis as detailed in Example 2.4. Each bar represents the relative frequency of each CAR candidate among the mix of the 4 CARs at the different steps of reactivation using CLL1 positive HL60 target cells. The relative frequency was calculated as per the following formula: Relative frequency=(# of analyzable reads for a given CAR/# of total analyzable reads)×100. Error bars correspond to triplicates ofthe time point. Although it appears that the four candidates behave similarly, the proportion of M2R2 and M26R2 tend to increase over M2QR3 and M26QR3, which means that R2 architecture would be more competitive over the QR3 architecture.

FIG. 31: Graph representation of the CAR⁺ T cells cytolytic capacities towards antigen presenting cells (HL60) assessed in a flow-based cytotoxicity assay with respect to the four CAR candidates M2-QR3, M26-QR3, M2-R2 and M26-R2. The cell viability was measured after a 4 hour coculture with CART cells at effector/target ratios set at 2.5:1, 5:1, 10:1 and 20:1 respectively.

FIG. 32: Graph representation of the CAR⁺ T cells cytolytic capacities towards antigen presenting cells (HL60) assessed in a flow-based cytotoxicity assay with respect to the four CAR candidates M2-QR3, M26-QR3, M2-R2 and M26-R2. The cell viability was measured after an overnight coculture with CAR T cells at effector/target ratios set at 0.25:1, 0.5:1, 1:1 and 2:1 respectively.

FIG. 33: CAR+ T cells IFN-gamma secretion capacities towards antigen presenting cells (HL60 vs. JEKO) assessed in an ELISA immunoassay as detailed in Example 2.4. The CART cells were cocultured for 24 hours at an effector:target ratio of 1:1(about 50000 antigen presenting cells). IFN-g secretion was measured using the Quantikine® ELISA Human IFN-γ Immunoassay KIT, R&D Systems.

### DETAILED DESCRIPTION

Provided herein are in vitro and in vivo screening assays for selecting a sub-population(s) of CAR immune cells that have a preferential capability to make immune cells proliferate in an antigen-dependent manner, wherein the sub-population(s) of CAR immune cells is selected from a population of immune cells endowed with a variety of CAR polynucleotides. In some embodiments, the methods are capable of predicting the activity of the CAR immune cells in vivo and can bypass costly and time consuming standard in vivo assays that are usually performed to screen or compare activity of different CAR constructs and check quality of CAR T cell clinical batches. In some embodiments, the screening assays enable the discrimination between CAR immune cells that show similar profiles with standard assays and short term (e.g., 4 hours) cytotoxicity assays.

In some other embodiments, the method enables to discriminate CARs bearing identical antibodies or antibodies directed against the same antigen or epitope, while displaying different structures. By different structures is meant that a variation exists between the different protein domains included in the CAR polypeptide, such as distinct transmembrane domains, hinges, linkers, co-activation domains, activation domains, domain interacting with other molecules, etc. This can be performed in view of determining which structures confer optimal potency to the immune cells. By “potency” is meant the overall immune activity of the cell that may confer therapeutic benefits, such as, for example, cytotoxicity, cytokine release against the target cells, more persistence in-vitro or in-vivo, cell duplication, less alloreactivity, reduced risk of GvHD (Graft versus Host Disease), less CRS (cytokine release syndrome), or less inflammation.

In some other embodiments, the invention enables to discriminate CARs having the same structure, while bearing different antibodies directed against the same or different antigens/epitopes. This can be performed, for instance, as part of a screening process, such as to select the most appropriate humanized versions of an antibody. According to this embodiment, the present invention encompasses a method for humanizing CARs, wherein mutations are introduced into a polynucleotide sequence encoding a CAR scaffold, especially into a non-human sequence (i.e. sequence encoding a polypeptide which is non-human), more especially into ScFv or murine origin, to produce humanized CAR variants. Immune cells are then endowed with such CAR variants and incubated repeatedly together with target cells until a dominant sub-population of immune cells is identified as per the invention, as resulting from antigen-dependent activated immune cells.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Unless specifically defined herein, all technical and scientific terms used have the same meaning as commonly understood by a skilled artisan in the fields of gene therapy, biochemistry, genetics, immunology, cancer and molecular biology. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

In one embodiment, the invention provides an in vitro method for selecting a candidate chimeric antigen receptor (CAR) polynucleotide to be expressed in immune cells for its preferential capability to make immune cells proliferate in an antigen-dependent manner, comprising
i) providing a population of immune cells endowed with a variety of CAR polynucleotides targeting the same antigen;
ii) incubating the population of CAR immune cells under i) with target cells expressing the same antigen for a period of time;
iii) adding an additional quantity of target cells to the incubated CAR immune cells and incubating for an additional period of time;
iv) optionally repeating step iii) one or more times;
v) detecting the presence of an enriched sub-population(s) of CAR immune cells by sequencing or amplifying the polynucleotides encoding the variety of CARs; and
vi) selecting the CAR polynucleotide expressed by said enriched sub-population of CAR immune cells.

In accordance with the invention, the method encompasses providing a population of immune cells endowed with a variety of CAR polynucleotides targeting the same antigen. The immune cells of the population are modified to express a variety of CARs encoded by the polynucleotides. In some embodiments, an individual cell generally expresses a single type of CAR encoded by a particular polynucleotide. An immune cell comprising a CAR can redirect immune cell specificity and reactivity toward a selected target exploiting ligand-binding domain properties. A CAR combines a binding domain against a component present on the target cell, for example an antibody-based specificity for a desired antigen (e.g., tumor antigen) with a T cell receptor-activating intracellular domain to generate a chimeric protein that exhibits a specific anti-target cellular immune activity. In some embodiments, the variety of CAR polynucleotides encodes different CAR architectures. For example, in some embodiments, the CAR architecture comprises single-chain or multi-chain CARs as disclosed in International Application No. PCT/US2013/058005 (International Application Pub. No. WO 2014/039523), which is incorporated herein by reference. In some embodiments, the CAR binding domain can comprise an extracellular single chain antibody (scFv) fused to the intracellular signaling domain of the T cell antigen receptor complex zeta chain (scFv:ζ) and have the ability, when expressed in T cells, to redirect antigen recognition based on the binding domain's specificity. In some embodiments, the CAR comprises an antigen binding domain (e.g., scFv), a signaling domain (e.g., CD3 zeta chain), and a co-stimulatory domain (e.g., CD28). In some embodiments, the CAR comprises an antigen binding domain (e.g., scFv), a signaling domain (e.g., CD3 zeta chain), and two co-stimulatory domains (e.g., CD28 and 4-1 BB). In some embodiments, chimeric scFv, which is formed of the VH and VL polypeptides and the specific epitope(s) may itself have different structures depending on the position of insertion of the epitope and the use of linkers. In some embodiments, the CAR polynucleotide comprises a humanized scFv.

Immune cells are generally endowed with CARs through introduction and heterologous expression into said cells of exogenous polynucleotide sequences encoding them. Various methods for introducing these exogenous coding sequences are available, among which the use of retroviral vectors, especially lentiviral vectors that integrate into cells genome upon transduction as described for instance by Scholler, J. et al. (Decade-Long Safety and Function of Retroviral-Modified Chimeric Antigen Receptor T Cells (2012) Science Translational Medicine 4(132):132). The CAR encoding polynucleotides can also be introduced under mRNA form by electroporation as described for instance by Boissel et al. (Transfection with mRNA for CD19 specific chimeric antigen receptor restores NK cell mediated killing of CLL cells (2009) Leukemia Research. 33(9):1255-59). According to a preferred embodiment, the CAR encoding polynucleotides are introduced into the genome by site directed integration at a predetermined locus by introduction or expression into the cell of a rare-cutting endonuclease, such as described by Eyquem J. et al. (Targeting a CAR to the TRAC locus with CRISPR/Cas9 enhances tumor rejection (2017) Nature 543:113-117). This integration can be performed by cloning the CAR encoding polynucleotides on rAAV6 vectors to be used as DNA template to perform the integration by homologous recombination (HR) of non-homologous end joining (NHEJ) integration.

This is particularly advantageous to integrate the CAR encoding polynucleotide at the TCR locus, when one is willing to inactivate T-cells receptor (TCR) to produce allogeneic T-cells having reduced alloreactivity, as formerly described in WO2013176915. The Immune cells may however be endowed with CARs by any further methods known in the art.

The term “antigen” is well understood in the art and includes substances which are generally immunogenic, i.e., immunogens, as well as antigenic epitopes. It will be appreciated that the use of any antigen is envisioned for use in the present invention and thus includes, but is not limited to, a self-antigen (whether normal or disease-related), an infectious antigen (e.g., a microbial antigen, viral antigen, etc.), or some other foreign antigen. In some embodiments, the antigen is from a cancer cell.

In some embodiments, the antigen is autologous to a subject. By autologous to the subject is meant that the antigen is obtained or derived from a subject. As non-limiting examples, the antigen can be from a cancer cell or tumor tissue obtained from a subject.

In some embodiments, the antigen can include, but is not limited to, 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvlll (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor—2), hTERT or hTRT (human telomerase reverse transcriptase), iCE (intestinal carboxyl esterase), IL-13R-a2 (interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidas e 2-a-Lfucosyltransferase), LeY (Lewis-Y antibody), L1 CAM (L1 cell adhesion molecule), MAGE (melanoma antigen), MAGE-A1 (Melanoma-associated antigen 1), mesothelin, Murine CMV infected cells, MART-1/Melan-A (melanoma antigen recognized by T cells-I/Melanoma antigen A), MC1 R (melanocortin 1 receptor), Myosin/m (myosin mutated), MUC1 (mucin 1), MUM-1 , -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1, -2, -3, 4 (synovial sarcoma X1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), or WT1 (Wilms' tumor gene). In some embodiments, the antigen targeted by the CAR immune cells is selected from common markers of liquid tumors, such as CD19, CD20, CD22, CD30, CD33, CD123, CD133, ROR1, CLL1, BCMA, κ light chain, CD138, or from common markers of solid tumors, such as CS1, HSP70, CD38, EGFRvIII, GD2, GD3, HER2, CD70, CEA, Mesothelin, Mucl, ROR1, PSMA. VEGFR2 and NKG2D ligands. In some preferred embodiments, the antigen is CD22. In some embodiments, the CAR polynucleotide comprises a nucleic acid sequence encoding a scFv anti-CD22 sequence comprising any one of SEQ ID NOS:1-5. In some embodiments, the variety of CARs encoded by the polynucleotides target the same epitope on the antigen. In some embodiments, the variety of CARs encoded by the polynucleotides target different epitopes on the antigen. In some embodiments, one or more of the CARs encoded by the polynucleotides target different epitopes and one or more of the CARs encoded by the polynucleotides target the same epitope on the antigen.

In some embodiments, the antigen is a viral antigen. In some embodiments, the viral antigen can include, but is not limited to, an Epstein-Barr virus (EBV) antigen (e.g., EBNA-1, EBNA-2, EBNA-3, LMP-1, LMP-2), a hepatitis A virus antigen (e.g., VP1 , VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gl, gJ, gK, gL, gM, UL20, UL32, US43, UL45, UL49A), a cytomegalovirus (CMV) viral antigen (e.g., glycoproteins gB, gC, gC, gE, gG, gH, gl, gJ, gK, gL, gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen.

In some embodiments, the antigen is associated with cells having an immune or inflammatory dysfunction. In some embodiments, the antigen includes, but is not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

The immune cells according to the present invention preferably refer to primary cells of hematopoietic origin functionally involved in the initiation and/or execution of innate and/or adaptative immune response.

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-Kl cells; HEK293 cells; Caco2 cells; U2-OS cells; NIH 3T3 cells; NSO cells; SP2 cells; CHO-S cells; DG44 cells; K-562 cells, U-937 cells; MRCS cells; IMR90 cells; Jurkat cells; HepG2 cells; HeLa cells; HT-1080 cells; HCT-116 cells; Hu-h7 cells; Huvec cells; Molt 4 cells. Primary cells are generally used in cell therapy as they are deemed more functional and less tumorigenic.

In general, primary immune cells are provided from donors or patients through a variety of methods known in the art, as for instance by leukapheresis techniques as reviewed by Schwartz J.et al. (Guidelines on the use of therapeutic apheresis in clinical practice-evidence-based approach from the Writing Committee of the American Society for Apheresis: the sixth special issue (2013) J Clin Apher. 28(3):145-284).

The primary immune cells according to the present invention can also be differentiated from stem cells, such as cord blood stem cells, progenitor cells, bone marrow stem cells, hematopoietic stem cells (HSC) and induced pluripotent stem cells (iPS).

According to a preferred aspect of the invention the engineered cells are primary immune cells, such as NK cells or T-cells, which are generally part of populations of cells that may involve different types of cells. In general, population deriving from patients or donors isolated by leukapheresis from PBMC (peripheral blood mononuclear cells).

The immune cells according to the present invention in some embodiments are T cells or NK cells obtained from a donor. In some embodiments, the T-cells can be derived from a stem cell or differentiated from iPS cell lines. In some embodiments, the stem cells can be adult stem cells, embryonic stem cells, more particularly non-human stem cells, cord blood stem cells, progenitor cells, bone marrow stem cells, totipotent stem cells or hematopoietic stem cells. Representative human stem cells are CD34+ cells. The immune cell can also be a dendritic cell, killer dendritic cell, a mast cell, a NK-cell, a B-cell, a macrophage or a T cell selected from the group consisting of inflammatory T-lymphocytes, cytotoxic T-lymphocytes, regulatory T-lymphocytes or helper T-lymphocytes. In another embodiment, the immune cell can be derived from the group consisting of CD4+ T-lymphocytes and CD8+ T-lymphocytes. Prior to expansion and genetic modification of the cells, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art can be used. In some embodiments, the immune cells are derived from a healthy donor.

In one of its plainest embodiments, the invention relates to a method where immune cells are endowed with different CARs, said cells being pooled and incubated together to discriminate, under predetermined growing conditions, which CARs are able to generate antigen-dependent populations.

According to one embodiment, the method of the invention comprises one or several of the following steps, wherein:
a) immune cells are transfected with exogenous polynucleotide sequences encoding a variety of CARs having different structures or different antigen binding domains,
b) the transfected immune cells are pooled together into an environment favorable to their growth, in-vitro or in-vivo, in the presence of a population of non-immune cells such as malignant cells, preferably ghost cells, bearing antigen that are expected to bind the CARs,
c) the immune cells incubated in said environment are sampled after a period of time to determine which CAR is able to promote a dominant sub-population(s).

In one embodiment, the invention provides an in vitro method for selecting a candidate chimeric antigen receptor (CAR) polynucleotide to be expressed in immune cells for its preferential capability to make immune cells proliferate in an antigen-dependent manner, comprising
i) providing a population of immune cells endowed with a variety of CAR polynucleotides targeting the same antigen;
ii) incubating the population of CAR immune cells under i) with target cells expressing the same antigen for a period of time;
iii) adding an additional quantity of target cells to the incubated CAR immune cells and incubating for an additional period of time;
iv) optionally repeating step iii) one or more times;
v) detecting the presence of an enriched sub-population(s) of CAR immune cells by sequencing, preferably by deep-sequencing, or amplifying the polynucleotides encoding the variety of CARs; and
vi) selecting the CAR polynucleotide expressed by said enriched sub-population of CAR immune cells.

In accordance with the invention, the methods provide for incubating the population of CAR immune cells with target cells expressing the same antigen for a period of time. The period of time is not particularly limiting. In some embodiments, the period of time of steps ii) and/or iii) of the in vitro method ranges from about 12 hours to about 120 hours. In some embodiments, the period of time of steps ii) and/or iii) of the in vitro method is about 16-36 hours. In some embodiments, the period of time of steps ii) and iii) of the in vitro method is about 16-30 hours. In some embodiments, the period of time of steps ii) and iii) of the in vitro method is about 20-24 hours. The CAR immune cells and target cells can be incubated in conventional media and is not particularly limiting.

In some embodiments, the CAR immune cells are T cells. In some embodiments, the methods described herein can include a step of stimulating the population of immune cells with one or more T cell stimulating agents to produce a population of activated T cells. Any combination of one or more suitable T cell stimulating agents may be used to produce a population of activated T cells including, but is not limited to, an antibody or functional fragment thereof which targets a T cell stimulatory or co-stimulatory molecule (e.g., anti-CD2 antibody, anti-CD3 antibody, anti-CD28 antibody, or functional fragments thereof), a T cell cytokine (e.g., any isolated, wildtype, or recombinant cytokines such as: interleukin 1 (IL-1), interleukin 2, (IL-2), interleukin 4 (IL-4), interleukin 5 (IL-5), interleukin 7 (IL-7), interleukin 15 (IL-15), tumor necrosis factor α (TNFα)), or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T cell stimulatory or co-stimulatory molecule.

In some embodiments, the CAR immune cells and target cells are incubated in X-Vivo-15 media (Lonza) supplemented by 35UI/ml 10% FBS for coculture. and kept in culture at 37° C. in the presence of 5% CO₂.

In some embodiments, the target cells are cancer cells. By “cancer” is meant the abnormal presence of cells which exhibit relatively autonomous growth, so that a cancer cell exhibits an aberrant growth phenotype characterized by a significant loss of cell proliferation control. Cancerous cells can be benign or malignant. Various types of cancer are known and the particular cancer cells are not limiting. A cancer cell, as used herein, includes not only a primary cancer cell, but also any cell derived from a cancer cell. This includes metastasized cancer cells, and in vitro cultures and cell lines derived from cancer cells. Cancer includes, but is not limited to, solid tumors and hematologic malignancies.

In some embodiments, the cancer is selected from Non-Hodgkin's lymphoma (NHL), diffuse large B cell lymphoma (DLBCL), small lymphocytic lymphoma (SLL/CLL), mantle cell lymphoma (MCL), follicular lymphoma, marginal zone lymphoma (MZL), extranodal (MALT lymphoma), nodal (Monocytoid B-cell lymphoma), splenic, diffuse large cell lymphoma, B cell chronic lymphocytic leukemia/lymphoma, Burkitt's lymphoma, lymphoblastic lymphoma, multiple myeloma, acute lymphoblastic leukemia

(ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous T cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myeloproliferative disorders, nasal cavity and paranasal sinus cancer, nasopharyngeal cancer, neuroblastoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sezary syndrome, small cell lung cancer, small intestine cancer, soft tissue sarcoma, squamous cell carcinoma, squamous neck cancer, stomach (gastric) cancer, supratentorial primitive neuroectodermal tumors, T cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenstrom macroglobulinemia, and Wilms Tumor.

According to preferred embodiments, the target cells, which are used in the present invention to selectively activate the CAR immune cells in an antigen dependent manner, are replication deficient. In some embodiments, the target cells have been irradiated to impair their ability to replicate. Using irradiated cells can be advantageous in some embodiments where using living cells can tend to bias the results of the competition because the cells keep growing and their concentration can sometimes get too high. In some embodiments, the cells are irradiated at a dose of about 20-100 Gy. In some embodiments, the cells are irradiated at a dose of 60 Gy. See, eg., Compact X-Ray Irradiation System-CellRad, Faxitron #2328A50149. In some embodiments, the target cells are NALM-16 cells that are CD22⁺.

The step of adding an additional quantity of target cells to the incubated CAR immune cells and incubating for an additional period of time can optionally be repeated one or more times. In some embodiments, the step is repeated from 1 to about 50 times. In some embodiments, the step is repeated 1 to about 35 times. In some embodiments, the step is repeated 3 to 5 times. In some embodiments, the step is repeated 1, 2, 3, 4, 5, 6, 7, 8, 9, 10, 11, 12, 13, 14 or 15 times.

The ratio of the CAR immune cells to the target cells is not particularly limiting. In some embodiments, the CAR immune cells are incubated with the target cells at a ratio of about 1:20 to about 20:1. In some embodiments, the CAR immune cells are incubated with the target cells at a ratio of about 1:2, about 1:4, about 1:8 or about 1:16. In some embodiments, the CAR immune cells are incubated with the target cells at a ratio of about 1:1.

In some embodiments, the additional quantity of target cells that is repeatedly added to the CAR immune cells is a substantially constant quantity of cells. In some embodiments, the additional quantity of target cells is from about 10⁵ to about 10⁷ cells. In some embodiments, the additional quantity of target cells is about 5×10⁵ cells. In some embodiments, the additional quantity of target cells is added to maintain a ratio of the CAR T cells to the target cells of about 1:20 to about 20:1. In some embodiments, the additional target cells are added to maintain a ratio of the CAR T cells to the target cells of about 1:1.

In some embodiments, the method further comprises assaying the quantity of target cells. In some embodiments, the quantity of target cells is assayed prior to adding an additional quantity of target cells to the incubated CAR immune cells. In some embodiments, the quantity of target cells is assayed so that it can be determined what quantity of target cells should be added to the incubated CAR immune cells. In some embodiments, the quantity of target cells is assayed about every 12-96 hours. In some embodiments, the quantity of target cells is assayed about every 24-48 hours. In some embodiments, the quantity of target cells is assayed once a day or every few days. In some embodiments, the quantity of target cells present is assayed during and/or after the incubating steps ii) and iii).

In some embodiments, the method further comprises assaying the quantity of CAR immune cells present one or more times. In some embodiments, the quantity of CAR immune cells is assayed about every 12-96 hours. In some embodiments, the quantity of CAR immune cells is assayed about every 24-48 hours. In some embodiments, the quantity of CAR immune cells present is assayed during and/or after the incubation steps ii) and iii).

The methods used to determine the quantity of CAR immune cells and/or target cells is not limiting. In some embodiments, the quantity of CAR immune cells and/or target cells can be determined by assaying for the presence of a detectable label. In some embodiments, the label is selected from the group consisting of a chromophore, a fluorophore, a fluorescent protein, a phosphorescent dye, a tandem dye, a particle, a hapten, an enzyme, a radioisotope and combinations thereof. In some embodiments, the label is an enzyme selected from the group consisting of a peroxidase, a phosphatase, a glycosidase, and a luciferase.

In some embodiments, the quantity of CAR immune cells and/or target cells is determined by flow cytometry and/or cell counting (e.g., LUNA™ Automated Cell Counter/Trypan). In some embodiments, the CAR immune cells are labeled with one or more antibodies. In some embodiments, the CAR immune cells are labeled with an anti-CD3 antibody. In some embodiments, the CAR immune cells are labeled with an anti-CD8 antibody.

In some embodiments, the activity of the CAR immune cells is assayed by assaying the cell culture supernatant to determine the concentration of interferon gamma one or more times. In some embodiments, the concentration of interferon gamma is assayed during and/or after the incubation steps ii) and iii).

In some embodiments, the method further comprises assaying the quantity of dead target cells present one or more times. In some embodiments, the quantity of dead target cells is assayed about every 12-96 hours. In some embodiments, the quantity of dead target cells is assayed about every 24-48 hours. In some embodiments, the quantity of dead target cells present is assayed during and/or after the incubating step.

In some embodiments, the method further comprises comparing any results obtained from the method using the CAR immune cells with results obtained from the method using one or more different samples of CAR immune cells.

The method provides detecting the presence of an enriched sub-population(s) of CAR immune cells by sequencing or amplifying the polynucleotides encoding the various types of CARs. In some embodiments, the enriched sub-population of CAR immune cells is detected by polymerase chain reaction (PCR). In some embodiments, the enriched sub-population of CAR immune cells is detected by deep sequencing analysis. Deep sequencing refers to sequencing a genomic region multiple times which allows for the detection of rare clonal types comprising as little as 1% or less of the original sample. In some embodiments, a single-chain variable fragment (scFv) region of the CAR immune cells is amplified and sequenced. In some embodiments, the region comprises about 100 to about 400 base pairs in length. In some embodiments, the region comprises about 400 base pairs in length. In some embodiments, the enriched sub-population of CAR immune cells is detected by PCR using a primer set that is specific for the enriched sub-population of CAR immune cells. In some embodiments, the enriched sub-population of CAR immune cells is detected by PCR using a first primer that is specific to the enriched sub-population of CAR immune cells and a second primer that is common to a variety of the CARs targeting the same antigen. In some embodiments, one or more nucleotide tags of identical sequence can be added to the CAR polynucleotides to enable the use of universal primers to amplify the CAR polynucleotides.

In some embodiments, the polynucleotides can be amplified or sequenced at more than one time point during the method. For example, samples of cells can be obtained throughout the course of the experiment, and the CAR polynucleotides can be amplified or sequenced to monitor enrichment of a sub-population of CAR immune cells over time. For example, in some embodiments, cell pellets can be harvested and analyzed every day or every few days. In some embodiments, cell pellets are harvested at day 0, day 4, day 7, and day 11 of the method for analysis.

In some embodiments, the polynucleotides that have been amplified are of identical length. In some embodiments, the polynucleotides that have been amplified are between about 100 and 400 base pairs in length. In some embodiments, the polynucleotides that have been amplified are about 100, 125, 150, 175, 200, 225, 250, 275, 300, 325, 350, 375, or 400 base pairs in length. In some embodiments, the polynucleotides of the variety of CARs that have been amplified differ in length by no more than about 50 nucleotides, by no more than about 40 nucleotides, by no more than about 30 nucleotides, by no more than about 25 nucleotides, by no more than about 20 nucleotides, by no more than about 15 nucleotides, by no more than about 10 nucleotides, by no more than about 9 nucleotides, by no more than about 9 nucleotides, by no more than about 8 nucleotides, by no more than about 7 nucleotides, by no more than about 6 nucleotides, by no more than about 5 nucleotides, by no more than about 4 nucleotides, by no more than about 3 nucleotides, by no more than about 2 nucleotides, or by no more than about 1 nucleotide.

In some embodiments, polynucleotides encoding scFv anti-CD22 sequences can be amplified using oligonucleotide primers comprising SEQ ID NO:6 and SEQ ID NO:7.

In some embodiments, the CAR immune cells can be engineered to acquire one or more additional attributes that can improve their therapeutic effectiveness. In some embodiments, the CAR immune cells are engineered to be allogenic. Methods of making allogenic T cells are described, for example, in International Application No. PCT/EP2016/051471 (WO 2016/120220), which is incorporated herein by reference. In some embodiments, the CAR immune cells are engineered to confer resistance to at least one immune suppressive drug, and/or chemotherapy agent, such as a purine analog. In some embodiments, the CAR immune cells can be engineered to comprise an inactivating mutation in one or more immune-checkpoint genes. In some embodiments, the CAR immune cells are engineered to harbor a suicide gene to help deplete the CAR immune cells in a subject. Methods of making immune cells resistant to an immune suppressive drug, chemotherapeutic agent or have an inactivating mutation in an immune checkpoint gene or harbor a suicide gene is disclosed, e.g., in U.S. Patent Application Pub. No.: 2016/0361359 A1, which is incorporated herein by reference.

In some embodiments, the CAR immune cells comprise allogenic T cells, which can be useful in allogeneic immunotherapy. In some embodiments, the allogenic T cells comprise an inactivating mutation in at least one gene encoding a T cell receptor (TCR) component. In some embodiments, the TCR is rendered nonfunctional in the cells by inactivating a TCR alpha gene and/or a TCR beta gene(s). In some embodiments, the TCR inactivation in allogeneic T cells avoids graft versus host disease (GvHD). By inactivating a gene, it is intended that the gene of interest is not expressed in a functional protein form.

In some embodiments, the CAR immune cells are resistant to one or more chemotherapeutic agents. In some embodiments, the method further comprises incubating the CAR immune cells and the target cells in the presence of the chemotherapeutic agent. In some embodiments, the chemotherapeutic agent is a purine analogue drug. In some embodiments, the purine analogue drug is clofarabine or fludarabine. In some embodiments, deoxycytidine kinase (dcK—EC 2.7.1.74) is inactivated in the CAR immune cells.

The term “chemotherapeutic agent” as used herein refers to a compound or a derivative thereof that can interact with a cancer cell, thereby reducing the proliferative status of the cell and/or killing the cell. Examples of chemotherapeutic agents include, but are not limited to, alkylating agents (e.g., cyclophosphamide, ifosamide), metabolic antagonists (e.g., purine nucleoside antimetabolite such as clofarabine, fludarabine or 2′-deoxyadenosine, methotrexate (MTX), 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, and the like. Such agents may further include, but are not limited to, the anti-cancer agents TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, or a therapeutic derivative of any thereof.

As used herein, a cell which is “resistant or tolerant” to an agent means a cell which has been genetically modified so that the cell proliferates in the presence of an amount of an agent that inhibits or prevents proliferation of a cell without the modification.

In some embodiments, drug resistance can be conferred to the CAR immune cell by the expression of at least one drug resistance gene. The drug resistance gene refers to a nucleic acid sequence that encodes “resistance” to an agent, such as a chemotherapeutic agent (e.g. methotrexate). In other words, the expression of the drug resistance gene in a cell permits proliferation of the cells in the presence of the agent to a greater extent than the proliferation of a corresponding cell without the drug resistance gene. A drug resistance gene of the invention can encode resistance to antimetabolite, methotrexate, vinblastine, cisplatin, alkylating agents, anthracyclines, cytotoxic antibiotics, anti-immunophilins, their analogs or derivatives, and the like.

Several drug resistance genes have been identified that can potentially be used to confer drug resistance to targeted cells (Takebe, N., S. C. Zhao, et al. (2001). Mol Ther 3(1): 88-96; Sugimoto, Y., S. Tsukahara, et al. (2003). J Gene Med 5(5): 366-76; Zielske, S. P., J. S. Reese, et al. (2003). J Clin Invest 112(10): 1561-70; Nivens, M. C, T. Felder, et al. (2004). Cancer Chemother Pharmacol 53(2): 107-15; Bardenheuer, W., K. Lehmberg, et al. (2005). Leukemia 19(12): 2281-8; Kushman, M. E., S. L. Kabler, et al. (2007). Carcinogenesis 28(1): 207-14).

One example of a drug resistance gene can be a mutant or modified form of Dihydrofolate reductase (DHFR). DHFR is an enzyme involved in regulating the amount of tetrahydrofolate in the cell and is essential to DNA synthesis. Folate analogs such as methotrexate (MTX) inhibit DHFR and are thus used as anti-neoplastic agents in clinic. Different mutant forms of DHFR which have increased resistance to inhibition by anti-folates used in therapy have been described. In some embodiments, the drug resistance gene can be a nucleic acid sequence encoding a mutant form of human wild type DHFR (GenBank: AAH71996.1) which comprises at least one mutation conferring resistance to an anti-folate treatment, such as methotrexate. In some embodiments, a mutant form of DHFR comprises at least one mutated amino acid at position G15, L22, F31 or F34, preferably at positions L22 or F31 (Schweitzer, B. I., A. P. Dicker, et al. (1990). Faseb J 4(8): 2441-52); International Application Pub. No. WO 94/24277; U.S. Pat. No. 6,642,043). In some embodiments, the DHFR mutant form comprises two mutated amino acids at position L22 and F31. Correspondence of amino acid positions described herein is frequently expressed in terms of the positions of the amino acids of the form of wild-type DHFR polypeptide. In some embodiments, the serine residue at position 15 is preferably replaced with a tryptophan residue. In some embodiments, the leucine residue at position 22 can be replaced with an amino acid which will disrupt binding of the mutant DHFR to antifolates, preferably with uncharged amino acid residues such as phenylalanine or tyrosine. In another embodiment, the phenylalanine residue at positions 31 or 34 can be replaced with a small hydrophilic amino acid such as alanine, serine or glycine.

Another example of drug resistance gene can also be a mutant or modified form of ionisine-5′-monophosphate dehydrogenase II (IMPDH2), a rate-limiting enzyme in the de novo synthesis of guanosine nucleotides. The mutant or modified form of IMPDH2 is a IMPDH inhibitor resistance gene. IMPDH inhibitors can be mycophenolic acid (MPA) or its prodrug mycophenolate mofetil (MMF). In some embodiments, the mutant IMPDH2 can comprise at least one, or at least two mutations in the MAP binding site of the wild type human IMPDH2 (NP 000875.2) that lead to a significantly increased resistance to IMPDH inhibitor. The mutations can be at positions T333 and/or S351 (Yam, P., M. Jensen, et al. (2006). Mol Ther 14(2): 236-44; Sangiolo, D., M. Lesnikova, et al. (2007). Gene Ther 14(21): 1549-5). In some embodiments, the threonine residue at position 333 is replaced with an isoleucine residue and the serine residue at position 351 is replaced with a tyrosine residue. Correspondence of amino acid positions described herein is frequently expressed in terms of the positions of the amino acids of the form of wild-type human IMPDH2 polypeptide.

Another drug resistance gene is the mutant form of calcineurin. Calcineurin (PP2B) is a ubiquitously expressed serine/threonine protein phosphatase that is involved in many biological processes and which is central to T cell activation. Calcineurin is a heterodimer composed of a catalytic subunit (CnA; three isoforms) and a regulatory subunit (CnB; two isoforms). After engagement of the T cell receptor, calcineurin dephosphorylates the transcription factor NFAT, allowing it to translocate to the nucleus and active key target gene such as IL2. FK506 in complex with FKBP12, or cyclosporine A (CsA) in complex with CyPA block NFAT access to calcineurin's active site, preventing its dephosphorylation and thereby inhibiting T cell activation (Brewin, J., C. Mancao, et al. (2009). Blood 114(23): 4792-803). The drug resistance gene can also be a nucleic acid sequence encoding a mutant form of calcineurin resistant to calcineurin inhibitor such as FK506 and/or CsA. In some embodiments, the mutant form can comprise at least one mutated amino acid of the wild type calcineurin heterodimer a at positions: V314, Y341, M347, T351, W352, L354, K360, preferably double mutations at positions T351 and L354 or V314 and Y341. In a particular embodiment, the valine residue at position 341 can be replaced with a lysine or an arginine residue, the tyrosine residue at position 341 can be replaced with a phenylalanine residue; the methionine at position 347 can be replaced with the glutamic acid, arginine or tryptophane residue; the threonine at position 351 can be replaced with the glutamic acid residue; the tryptophane residue at position 352 can be replaced with a cysteine, glutamic acid or alanine residue, the serine at position 353 can be replaced with the histidine or asparagines residue, the leucine at position 354 can be replaced with an alanine residue; the lysine at position 360 can be replaced with an alanine or phenylalanine residue. Correspondence of amino acid positions described herein is frequently expressed in terms of the positions of the amino acids of the form of wild-type human calcineurin heterodimer (GenBank: ACX34092.1). In another embodiment, the mutant form can comprise at least one mutated amino acid of the wild type calcineurin heterodimer b at positions: V120, N123, L124 or K125, preferably double mutations at positions L124 and K125. In a particular embodiment, the valine at position 120 can be replaced with a serine, an aspartic acid, phenylalanine or leucine residue; the asparagine at position 123 can be replaced with a tryptophan, lysine, phenylalanine, arginine, histidine or serine; the leucine at position 124 can be replaced with a threonine residue; the lysine at position 125 can be replaced with an alanine, a glutamic acid, tryptophan, or two residues such as leucine-arginine or isoleucine-glutamic acid can be added after the lysine at position 125. Correspondence of amino acid positions described herein is frequently expressed in terms of the positions of the amino acids of the form of wild-type human calcineurin heterodimer b polypeptide (GenBank: ACX34095.1).

Another drug resistance gene is 0(6)-methylguanine methyltransferase (MGMT) encoding human alkyl guanine transferase (hAGT). AGT is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze, R., C. Kurpad, et al. (1999). J Pharmacol Exp Ther 290(3): 1467-74). In a particular embodiment, AGT mutant form can comprise a mutated amino acid of the wild type AGT position P140 (UniProtKB: P16455). In some embodiments, the proline at position 140 is replaced with a lysine residue.

Another drug resistance gene can be multidrug resistance protein 1 (MDR1) gene. This gene encodes a membrane glycoprotein, known as P-glycoprotein (P-GP) involved in the transport of metabolic byproducts across the cell membrane. The P-GP protein displays broad specificity towards several structurally unrelated chemotherapy agents. Thus, drug resistance can be conferred to cells by the expression of nucleic acid sequence that encodes MD-1 (NP 000918).

A useful drug resistance gene can also be cytotoxic antibiotics, such as the ble gene or mcrA gene. Ectopic expression of ble or mcrA in an immune cell gives a selective advantage when exposed to the chemotherapeutic agent, respectively the bleomycine or the mitomycin C.

In another embodiment, drug resistance can be conferred to the immune cell by the inactivation of a drug sensitizing gene. In some embodiments, the drug sensitizing gene which can be inactivated to confer drug resistance to the immune cell is the human deoxycytidine kinase (dCK) gene. This enzyme is required for the phosphorylation of the deoxyribonucleosides deoxycytidine (dC), deoxyguanosine (dG) and deoxyadenosine (dA). Purine nucleotide analogs (PNAs) are metabolized by dCK into mono-, di- and tri-phosphate PNA. Their triphosphate forms and particularly clofarabine triphosphate compete with ATP for DNA synthesis, acts as proapoptotic agent and are potent inhibitors of ribonucleotide reductase (RNR) which is involved in trinucleotide production.

In some embodiments, dCK inactivation in immune cells confers resistance to purine nucleoside analogs (PNAs) such as clofarabine and fludarabine.

In some embodiments, the dCK inactivation in immune cells is combined with an inactivation of TCR genes rendering these double knocked out (KO) T cells both resistant to drug such as clofarabine and allogeneic. This feature is particularly useful for a therapeutic goal, allowing “off-the-shelf” allogeneic cells for immunotherapy in conjunction with chemotherapy to treat patients with cancer. This double KO inactivation dCK/TCR can be performed simultaneously or sequentially.

Another example of enzyme which can be inactivated is human hypoxanthine-guanine phosphoribosyl transferase (HPRT) gene (Genbank: M26434.1). In some embodiments, HPRT can be inactivated in engineered immune cells to confer resistance to a cytostatic metabolite, the 6-thioguanine (6TG) which is converted by HPRT to cytotoxic thioguanine nucleotide and which is currently used to treat patients with cancer, in particular leukemias (Hacke, K., J. A. Treger, et al. (2013). Transplant Proc 45(5): 2040-4). Guanines analogs are metabolized by HPRT transferase that catalyzes addition of phosphoribosyl moiety and enables the formation of TGMP. Guanine analogues including 6 mercapthopurine (6MP) and 6 thioguanine (6TG) are usually used as lymphodepleting drugs to treat ALL. They are metabolized by HPRT (hypoxanthine phosphoribosyl transferase that catalyzes addition of phosphoribosyl moiety and enables formation TGMP. Their subsequent phosphorylations lead to the formation of their triphosphorylated forms that are eventually integrated into DNA. Once incorporated into DNA, thio GTP impairs fidelity of DNA replication via its thiolate groupment and generates random point mutations that are highly deleterious for cell integrity.

In another embodiment, inactivation of CD3 normally expressed at the surface of the T cell can confer resistance to anti-CD3 antibodies such as teplizumab.

In some embodiments, the immune cells are engineered to be resistant to multiple chemotherapeutic agents. In some embodiments, multiple drug resistance can be conferred by expressing more than one drug resistance gene and/or by inactivating more than one drug sensitizing gene. In some embodiments, multiple drug resistance can be conferred by expressing at least one drug resistance gene and inactivating at least one drug sensitizing gene. In some embodiments, multiple drug resistance can be conferred by expressing at least one drug resistance gene such as mutant form of DHFR, mutant form of IMPDH2, mutant form of calcineurin, mutant form of MGMT, the ble gene, and the mcrA gene and inactivating at least one drug sensitizing gene such as HPRT gene. In one embodiment, multiple drug resistance can be conferred by inactivating HPRT gene and expressing a mutant form of DHFR; or by inactivating HPRT gene and expressing a mutant form of IMPDH2; or by inactivating HPRT gene and expressing a mutant form of calcineurin; by inactivating HPRT gene and expressing a mutant form of MGMT; by inactivating HPRT gene and expressing the ble gene; by inactivating HPRT gene and expressing the mcrA gene.

In another embodiment, the immune cells can be modified to make them resistant to an immunosuppressive drug. For example, allogeneic cells can be rapidly rejected by the host immune system. Thus, in some embodiments, to prevent rejection of allogeneic cells, the host's immune system can be suppressed to some extent. However, in the case of adoptive immunotherapy, the use of immunosuppressive drugs can also have a detrimental effect on the introduced therapeutic immune cells. Therefore, in some embodiments, the introduced cells can be made resistant to the immunosuppressive treatment. In some embodiments, this can be done by inactivating at least one gene encoding a target for an immunosuppressive agent. An immunosuppressive agent is an agent that suppresses immune function by one of several mechanisms of action. In other words, an immunosuppressive agent is a role played by a compound which is exhibited by a capability to diminish the extent of an immune response. In some embodiments, the methods confer immunosuppressive resistance to immune cells for immunotherapy by inactivating the target of the immunosuppressive agent in immune cells. As nonlimiting examples, targets for immunosuppressive agent can be a receptor for an immunosuppressive agent such as: CD52, glucocorticoid receptor, a FKBP family gene member and a cyclophilin family gene member.

In another embodiment, the CAR immune cells comprise an inactivating mutation in one or more immune-checkpoint genes. T cell-mediated immunity includes multiple sequential steps involving the clonal selection of antigen specific cells, their activation and proliferation in secondary lymphoid tissue, their trafficking to sites of antigen and inflammation, the execution of direct effector function and the provision of help (through cytokines and membrane ligands) for a multitude of effector immune cells. Each of these steps is regulated by counterbalancing stimulatory and inhibitory signal that fine-tune the response. It will be understood by those of ordinary skill in the art, that the term “immune checkpoints” means a group of molecules expressed by T cells. These molecules effectively serve as “brakes” to down-modulate or inhibit an immune response. Immune checkpoint molecules include, but are not limited to Programmed Death 1 (PD-1, also known as PDCD1 or CD279, accession number: NM_005018), Cytotoxic T-Lymphocyte Antigen 4 (CTLA-4, also known as CD152, GenBank accession number AF414120.1), LAG 3 (also known as CD223, accession number: NM_002286.5), Tim3 (also known as HAVCR2, GenBank accession number: JX049979.1), BTLA (also known as CD272, accession number: NM_181780.3), BY55 (also known as CD160, GenBank accession number: CR541888.1), TIGIT (also known as VSTM3, accession number: NM_173799), LAIR1 (also known as CD305, GenBank accession number: CR542051.1), SIGLEC10 (GeneBank accession number: AY358337.1), 2B4 (also known as CD244, accession number: NM_001166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3 which directly inhibit immune cells. For example, CTLA-4 is a cell-surface protein expressed on certain CD4 and CD8 T cells; when engaged by its ligands (B7-1 and B7-2) on antigen presenting cells, T cell activation and effector function are inhibited. In some embodiments, the CAR immune cells are modified by inactivating at least one protein involved in the immune check-point, in particular PD-1 and/or CTLA-4.

In another embodiment, the CAR immune cells comprise an inactivating mutation in CD52 to make them resistant to Alemtuzumab.

In some embodiments, the CAR immune cells have been engineered to express a suicide gene. Since engineered immune cells can expand and persist for years after administration, it is desirable in some embodiments to include a safety mechanism to allow selective deletion of administrated immune cells. Thus, in some embodiments, the method of the invention can comprise the transformation of the immune cells with a recombinant suicide gene. The recombinant suicide gene can be used to reduce the risk of direct toxicity and/or uncontrolled proliferation of the immune cells once administrated in a subject. Suicide genes can enable selective deletion of transformed cells in vivo. The “suicide gene” can be a nucleic acid coding for a product, wherein the product causes cell death by itself or in the presence of other compounds. A representative example of such a suicide gene is one which codes for thymidine kinase of herpes simplex virus. Additional examples are thymidine kinase of varicella zoster virus and the bacterial gene cytosine deaminase which can convert 5-fluorocytosine to the highly toxic compound 5-fluorouracil. Suicide genes also include as nonlimiting examples caspase-9 or caspase-8 or cytosine deaminase. Caspase-9 can be activated using a specific chemical inducer of dimerization (CID). Suicide genes can also be polypeptides that are expressed at the surface of the cell and can make the cells sensitive to therapeutic monoclonal antibodies. As used herein “prodrug” means any compound useful in the methods of the present invention that can be converted to a toxic product. The prodrug can be converted to a toxic product by the gene product of the suicide gene in some embodiments. A representative example of such a prodrug is ganciclovir which is converted in vivo to a toxic compound by HSV-thymidine kinase. The ganciclovir derivative subsequently is toxic to tumor cells. Other representative examples of prodrugs include acyclovir, FIAU [1-2-deoxy-2-fluoro-(3-D-arabinofuranosyl)-5-iodouracil], 6-methoxypurine arabinoside for VZV-TK, and 5-fluorocytosine for cytosine deaminase. In some embodiments, the CAR immune cells have been engineered to express RQR8 protein and can be depleted by administration of Rituximab as described in WO WO2013153391.

The immune cells can be engineered to express the CAR and acquire the one or more additional attributes using known techniques and methods and is not limiting. Polypeptides can be expressed in the cell as a result of the introduction of polynucleotides encoding said polypeptides into the cell. Alternatively, said polypeptides could be produced outside the cell and then introduced thereto. Methods for introducing a polynucleotide construct into cells are known in the art and include as nonlimiting examples stable transformation methods wherein the polynucleotide construct is integrated into the genome of the cell, transient transformation methods wherein the polynucleotide construct is not integrated into the genome of the cell and virus mediated methods. The polynucleotides can be introduced into a cell by, for example, recombinant viral vectors (e.g. retroviruses, adenoviruses), liposome and the like. For example, transient transformation methods include, for example microinjection, electroporation or particle bombardment. The polynucleotides can be included in vectors, more particularly plasmids or virus, in view of being expressed in cells. The plasmid vector can comprise a selection marker which provides for identification and/or selection of cells which received said vector. Different transgenes can be included in one vector.

In some embodiments, the immune cells are transduced by a viral vector encoding the polypeptide of interest. The viral vector which can be used to transduce the cells is not limiting. In some embodiments, the viral vector will typically comprise a highly attenuated, non-replicative virus. Viral vectors include, but are not limited to, DNA viral vectors such as those based on adenoviruses, herpes simplex virus, avian viruses, such as Newcastle disease virus, poxviruses such as vaccinia virus, and parvoviruses, including adeno-associated virus; and RNA viral vectors, including, but not limited to, the retroviral vectors. Vaccinia vectors and methods useful in immunization protocols are described in U.S. Pat. No. 4,722,848. Retroviral vectors include lentiviruses such as human immunodeficiency virus. Naldini et al. (1996) Science 272:263-267. Replication-defective retroviral vectors harboring a nucleotide sequence of interest as part of the retroviral genome can be used. Such vectors have been described in detail. (Miller, et al. (1990) Mol. Cell. Biol. 10:4239; Kolberg, R. (1992) J. NIH Res. 4:43; Cornetta, et al. (1991) Hum. Gene Therapy 2:215).

Adenovirus and adeno-associated virus vectors may be produced according to methods already taught in the art. (See, e.g., Karlsson, et al. (1986) EMBO 5:2377; Carter (1992) Current Opinion in Biotechnology 3:533-539; Muzcyzka (1992) Current Top. Microbiol. Immunol. 158:97-129; Gene Targeting: A Practical Approach (1992) ed. A. L. Joyner, Oxford University Press, NY). Several different approaches are feasible.

Alpha virus vectors, such as Venezuelan Equine Encephalitis (VEE) virus, Semliki Forest virus (SFV) and Sindbis virus vectors, can be used for efficient gene delivery. Replication-deficient vectors are available.

In some embodiments, the viral vector is a retrovirus/lentivirus, adenovirus, adeno-associated virus, alpha virus, vaccinia virus or a herpes simplex virus. In some embodiments, the viral vector is a lentiviral vector.

In another embodiment, polynucleotides encoding polypeptides according to the present invention can be mRNA which are introduced directly into the cells, for example by electroporation. In some embodiments, mRNA is introduced by electroporation into T cells. In some embodiments, electroporation can be achieved using cytoPulse technology to transiently permeabilize living cells for delivery of material into the cells (see, e.g., U.S. Pat. No. 6,010,613 and International Application Publication No. WO 2004/083379).

In some embodiments, the cells are modified by a rare-cutting endonuclease that specifically catalyzes cleavage in a target gene. In some embodiments, the rare-cutting endonuclease can be a meganuclease, a Zinc finger nuclease, CRISPR/Cas9 nuclease, a TALE-nuclease. In one embodiment, the rare-cutting endonuclease is a TALE-nuclease. By TALE-nuclease is intended a fusion protein consisting of a DNA-binding domain derived from a Transcription Activator Like Effector (TALE) and one nuclease catalytic domain to cleave a nucleic acid target sequence.

The methods described above can be adapted and used in vivo. For example, in one embodiment, the invention provides an in vivo method for selecting a candidate CAR polynucleotide to be expressed in immune cells for its preferential capability to make immune cells proliferate in an antigen-dependent manner, comprising
i) providing a population of immune cells endowed with a variety of CAR polynucleotides targeting the same antigen;
ii) administering the population of immune cells to a subject having target cells comprising the antigen, wherein a CAR immune cell sub-population that exhibits a preferential capability to proliferate in an antigen-dependent manner becomes enriched in the population of CAR immune cells;
iii) detecting the presence of the enriched sub-population of CAR immune cells; and
iv) selecting the CAR polynucleotide expressed by said enriched sub-population of CAR immune cells.

The “subject” can refer to any bird, fish, reptile, amphibian, or mammal. In some embodiments, the subject is a mammal. In some embodiments, the subject is selected from a human, mouse, or a rat. In some embodiments, the subject is a mouse.

In some embodiments, the subject has cancer, as described herein. In some embodiments, the subject has a solid tumor. In some embodiments, the subject has a liquid tumor. In some embodiments, the in vivo method comprises administering additional target cells to the subject one or more times. In some embodiments, the additional target cells are administered 1, 2, 3, 4, 5, 6, 7, 8, 9, or 10 or more times to the subject. In some embodiments, additional target cells are administered once a day, every few days, every week, every two weeks, every 3 weeks, or about every month.

The enriched sub-population of CAR immune cells is detected after a period of time has elapsed since the initial administration step. In some embodiments, the method comprises detecting the presence of an enriched sub-population(s) of CAR immune cells by sequencing or amplifying the polynucleotides encoding the variety of CARs. In some embodiments, the enriched sub-population of CAR immune cells is detected about 10-120 days after administering the population of CAR immune cells to the subject. In some embodiments, the CAR immune cells can be monitored and detected throughout the experiment to monitor enrichment of a sub-population(s). For example, samples of cells can be obtained throughout the course of the experiment, and the CAR polynucleotides can be amplified or sequenced to monitor enrichment of a sub-population of CAR immune cells over time. For example, in some embodiments, cells can be isolated every day, every few days, every week, every few weeks or months to monitor enrichment of the CAR immune cell sub-population(s).

The CAR immune cells obtainable and selected according to the present methods can be used in adoptive cell immunotherapy. In some embodiments, the CAR immune cells can be used for treating cancer, infections or auto-immune disease in a patient in need thereof The treatment can be ameliorating, curative or prophylactic.

Cancers that may be treated include tumors that are not vascularized, or not yet substantially vascularized, as well as vascularized tumors. The cancers may comprise nonsolid tumors (such as hematological tumors, for example, leukemias and lymphomas) or may comprise solid tumors as described herein. In some embodiments about 1×10⁸ to 1×10¹⁰ CAR immune cells are administered per injection. In some embodiments, about 5×10⁹ cells are administered per injection.

Application of the teachings of the present invention to a specific problem is within the capabilities of one having ordinary skill in the art in light of the teaching contained herein. Examples of the compositions and methods of the invention appear in the following non-limiting Example.

### EXAMPLES

### Example 1. In Vitro Selection of CD22 CAR T Cells

The aim of this new strategy is to identify the best candidates among a pool of different CAR-T cells by combining an in vitro experiment such as an antigen dependent proliferation with a bioinformatic tool such as deep sequencing analysis.

PBMCs were thawed at day 0, activated using Transact human T activator CD3/CD28 beads at day 1. 3 days after their activation (day 4), 1 million T cells were transduced or not using a CD22 tool CAR and different candidates at a MOI of 5. Cells were then immediately diluted in X-Vivo-15 media supplemented by 20 ng/ml IL-2 and 5% CTS™ Immune Cell SR and diluted at 1×10⁶ cells/ml and kept in culture at 37° C. in the presence of5% CO₂. T cells were grown for 18 days, until the end of a classical process. At the end of the process, a pool of CD22 CAR transduced-T cells was co-cultured with target cell lines at an Effector:Target ratio of 1:1. So that the CAR-T cell expansion is only the consequence of a contact between CAR-T cells and positive target cell lines, this co-culture is performed in the presence of low doses of IL-2 (2 ng/mL). NALM-16 target cells were used as CD22⁺ cells while Sup-T1 were used as control CD22⁻ cells.

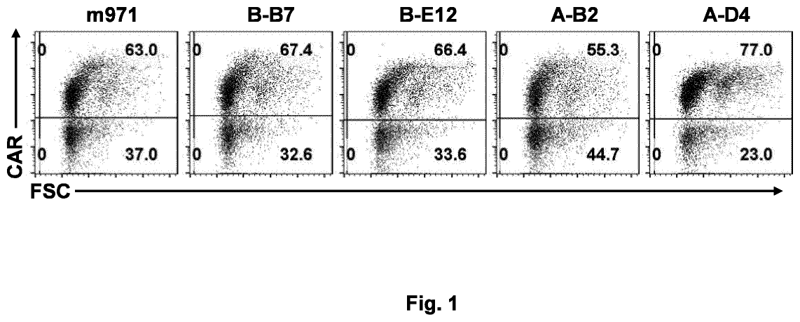

Five different CD22 CAR transduced-T cells with equivalent efficiencies of transduction and comparable frequencies of CAR expression overtime were selected (FIG. 1). 14 days post-transduction, about 3×10⁶ T cells from each group were centrifuged for 5 min at 300 g and resuspended in 1.5 mL of X-Vivo-15 media supplemented by 2 ng/ml IL-2 and 10% FBS. Cell densities were then determined using LUNA™ Automated Cell Counter/Trypan method. Then, the 5 types of CD22 transduced-T cells were mixed together using an equimolar ratio at 0.5×10⁶ cells/mL in X-VIVO™ 15, FBS 10%, IL-2 2 ng/mL culture media.

In parallel, 10 mL of positive (NALM-16, CD22⁺) and negative control (SupT1, CD22⁻) target cells were irradiated in T25 flask at 60Gy using Compact X-Ray Irradiation System-CellRad, Faxitron #2328A50149. Cells were then centrifuged at 1500 rpm for 5 mn and resuspended at 0.5·10⁶ cells/mL in X-VIVO™ 15, FBS 10%, IL-2 2 ng/mL culture media.

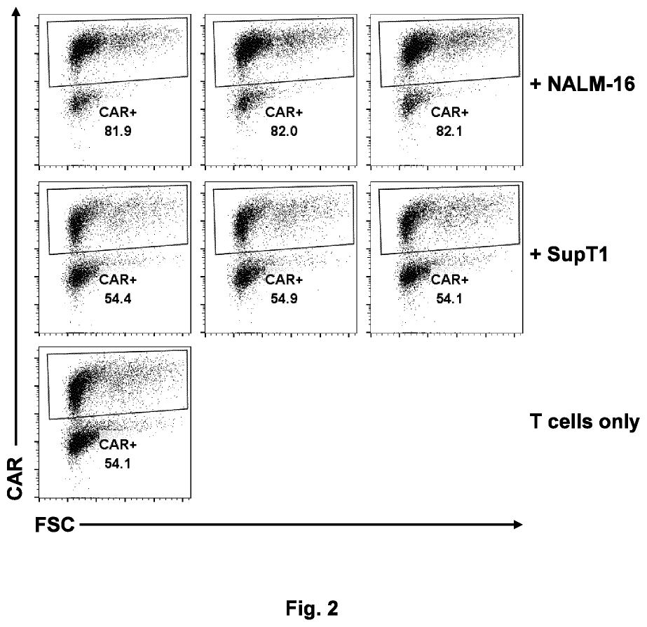

CD22 transduced-T cells were then cocultured with irradiated NALM-1 6, CD22⁺ or SupT1, CD22⁻ target cells (0.5·10⁶ cells from an equimolar mix of CD22 transduced-T cells+0.5·10⁶ target cells) in 2 mL of X-VIVO™ 15, FBS 10%, Il-2 2 ng/mL culture media in 24 well-plates (each condition was performed in triplicates). The same mix of CD22 transduced T cells, NALM-16 and SupT1 cells cultured alone were used as controls (one well per condition). This first coculture corresponds to a new day 0 of this serial CAR selection experiment. At this time point, 1×10⁶ cells from each group of CD22 transduced-T cells were harvested and cell pelleted, 3×10⁶ of irradiated NALM-16 or irradiated SupT1 cells were harvested and cell pelleted, as well as 3×10⁶ of mixed CD22 transduced-T cells. 4 days later, cell densities were determined using LUNA™ Automated Cell Counter/Trypan method in each well of the coculture. At day 4, more than 80% of the mix of CD22 transduced-T cells were CAR⁺ after reactivation by positive (NALM-16, CD22⁺) target cells (FIG. 2). When cells were cocultured with negative (SupT1, CD22⁻) target cells the frequency of CAR⁺ cells is stable and reaches 50% among CD3⁺ viable T cells. One more reactivation step was then performed at the same E:T 1:1 ratio as previously described. The mix of CAR-T cells was cocultured with irradiated NALM-16, CD22⁺ or SupT1, CD22⁻ target cells (0.5·10⁶ cells of CAR-T cells+0.5·10⁶ target cells) in 2 mL of X-VIVO™ 15, FBS 10%, Il-2 2 ng/mL culture media in 24 well-plates (each condition, meaning each triplicate from the previous step, was performed in duplicates). Before reactivation, 1×10⁶ cells from each group of CD22 transduced-T cells cocultured either with positive (NALM-16, CD22⁺) or with negative (SupT1, CD22⁻) target cells were harvested and cell pelleted. At this time point (d4), 1×10⁶ of T cells alone (mix) were also harvested and cell pelleted as control.

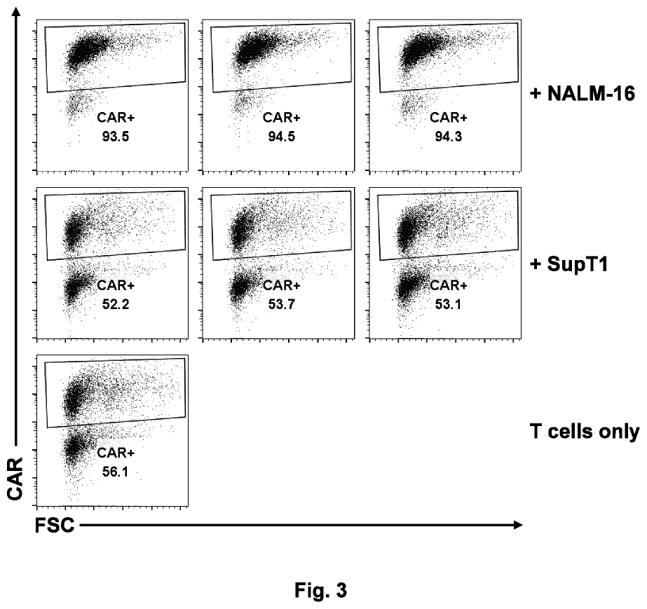

3 days later, cell densities were determined using LUNA™ Automated Cell Counter/Trypan method in each well of the coculture (duplicates were pooled, but each triplicate from the first step remained independent). At day 7, more than 90% of the mix of CD22 transduced-T cells were CAR⁺ after reactivation by positive (NALM-16, CD22⁺) target cells (FIG. 3). When cells were cocultured with negative (SupT1, CD22⁻) target cells the frequency of CAR⁺ cells is stable (50% among CD3⁺ viable T cells). At d7, 1×10⁶ cells from each group of CD22 transduced-T cells cocultured either with positive (NALM-16, CD22⁺) or with negative (SupT1, CD22⁻) target cells were harvested and cell pelleted. Cell viability being a bit low especially for CAR-T cells cocultured with SupT1 target cells, cells were seeded in 24 well plates at a cell density of 1×10⁶ cells/mL for one additional day. At d7, neither target cells (positive and negative) nor T cells alone were kept in culture because of low viability and small number of cells left. Before reactivation, 1×10⁶ cells from each group of CD22 transduced-T cells cocultured either with positive (NALM-16, CD22⁺) or with negative (SupT1, CD22⁻) target cells were harvested and cell pelleted. The following day (d8), cell viability for CAR-T cells that were reactivated with positive (NALM-16, CD22⁺) target cells was about 70% while cell viability for CAR-T cells that were cocultured with negative (SupT1, CD22⁻) target cells remained pretty low (around 40 to 50%). CAR-T cells were then reactivated at the same E:T 1:1 ratio as previously described. The mix of CAR-T cells was cocultured with irradiated NALM-16, CD22⁺ or SupT1, CD22⁻ target cells (0.5·10⁶ cells of CAR-T cells+0.5·10⁶ target cells) in 2 mL of X-VIVO™ 15, FBS 10%, Il-2 2 ng/mL culture media in 24 well-plates (one well per condition).

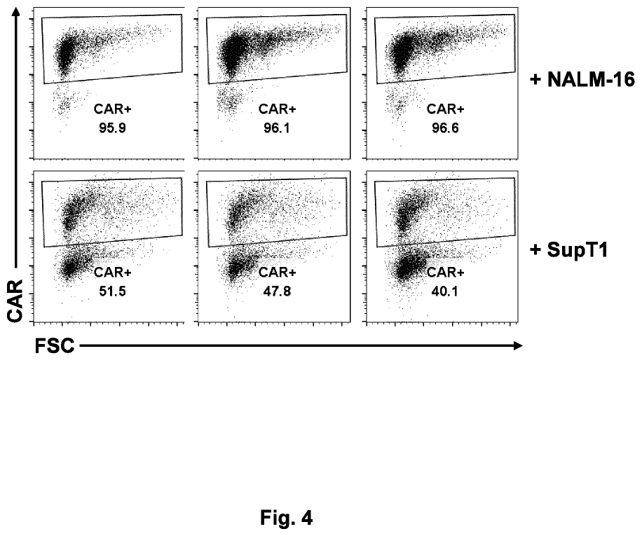

3 days later (d1), cell densities were determined using LUNA™ Automated Cell Counter/Trypan method in each well of the coculture. At day 11, 3 days after the third reactivation, more than 90% of the mix of CD22 transduced-T cells was CAR⁺ after reactivation by positive (NALM-16, CD22⁺) target cells (FIG. 4). When cells were cocultured with negative (SupT1, CD22⁻) target cells the frequency of CAR⁺ cells is stable (50% among CD3⁺ viable T cells). At d11, 1×10⁶ cells from each group of CD22 transduced-T cells cocultured either with positive (NALM-16, CD22⁺) or with negative (SupT1, CD22⁻) target cells were harvested and cell pelleted. 1×10⁶ cells from each group of CD22 transduced-T cells cocultured either with positive (NALM-16, CD22⁺) or with negative (SupT1, CD22⁻) target cells were harvested and cell pelleted.

In this whole process, cell pellets were harvested at d0, d4, d7 and d11. gDNA was extracted from all the samples using the DNeasy Blood & Tissue Kit following the manufacturer's instructions (QIAGEN, Cat No./ID 69506).

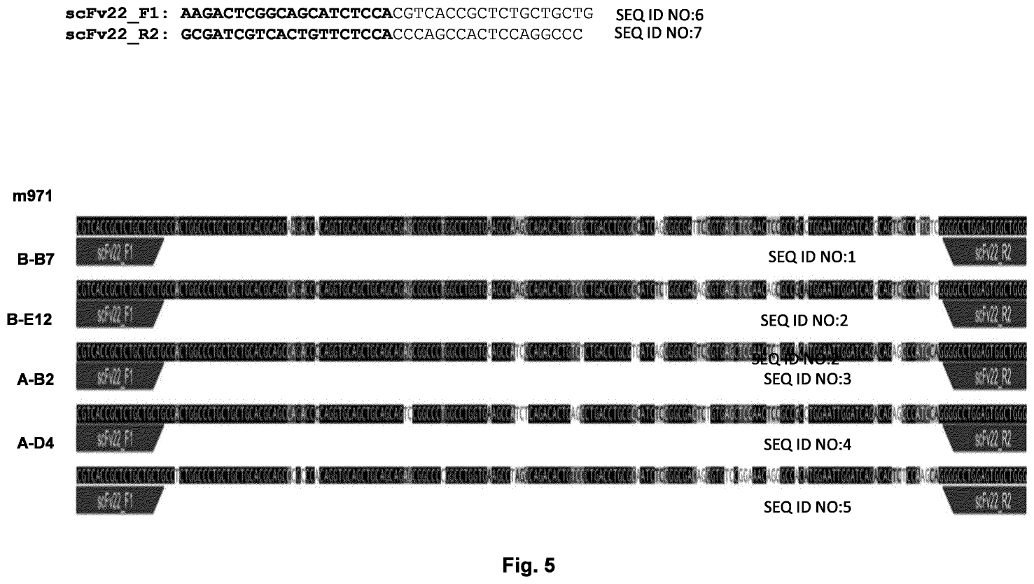

A set of primers was designed so as to be able to discriminate the different CARs by deep sequencing analysis. The CD22 CARs studied show many differences that would be detectable by deep sequencing. Even though only few regions are conserved between the scFv of the 5 CARs tested, we were able to select a set of primers common between them. The forward primer was located in the signal peptide which is exactly the same in all the constructions. The reverse primer was located in a short but conserved region about 200 bp away (FIG. 5). Then, PCR amplification was performed for each sample. The level of amplification was then assessed by migration on agarose gel. Positive samples, as defined by the presence of a band on 1% agarose gel, were purified using AMPure purification (Agencourt® AMPure® XP; Protocol 000387v001) following the manufacturer's instructions. As expected, no amplification was detected for gDNA samples corresponding to NALM-16 or SupT1 irradiated cells alone or to non-transduced T cells. DNA concentration was determined using the Quant-iT™ PicoGreen® dsDNA Assay Kit (Invitrogen P11496) and samples were sent for Illumina sequencing to an external platform at ICM. The nucleic acid and amino acid sequences of the CD22 CARs are shown below.

TABLE 1
Nucleic acid and amino acid sequences of the CD22 CARs.

| Name of the sequence | Nucleic Acid/Amino Acid Sequence |
|---|---|
| Oligo scFv22_F1 | AAGACTCGGCAGCATCTCCACGTCACCGCTCTGCTGCTG |
| | (SEQ ID NO: 6) |
| Oligo scFv22_R2 | GCGATCGTCACTGTTCTCCACCCAGCCACTCCAGGCCC |
| | (SEQ ID NO: 7) |
| pCLS29962; B-B7 | Nucleic acid: |
| | ATGGCTCTGCCCGTCACCGCTCTGCTGCTGCCACTGGCCCTG |
| | CTGCTGCACGCAGCCAGACCCCAGGTGCAGCTGCAGCAGAG |
| | CGGCCCTGGCCTGGTGGAGCCAAGCCAGACACTGTCCCTGA |
| | CCTGCGCCATCTCTGGCGACAGCGTGAGCTCCAACAGCGCC |
| | GCATGGAATTGGATCAGGCAGTCCCCATCTCGGGGCCTGGA |
| | GTGGCTGGGCAGAACATACTATAGGTCCACCTGGTACAACG |
| | ACTATGCCGGCTCCGTGAAGTCTCGCATCACAATCAACCCC |
| | GATACCAGCAAGAATCAGTTCTCCCTGCAGCTGACATCTGT |
| | GACCCCTGAGGACACAGCCGTGTACTATTGCACCAGAAGCA |
| | GGCACAATACATTTCGGGGAATGGACGTGTGGGGACAGGG |
| | CACACTGGTGACCGTGAGCGGAGGAGGAGGATCCGGCGGA |
| | GGAGGCTCTGGCGGCGGCGGCAGCGACATCCAGCTGACCC |
| | AGTCCCCTTCTAGCCTGAGCGCCTCCGTGGGCGATAGAGTG |
| | ACAATCACCTGTAGGGCCTCTCAGAGCATCTCCTCTTACCTG |
| | AACTGGTATCAGCAGAAGCCCGGCAAGGCCCCTAAGCTGCT |
| | GATCTACGCAGCAAGCTCCCTGCAGTCTGGAGTGCCAAGCA |
| | GATTCTCCGGCTCTGGCAGCGGCACCGACTTTACACTGACC |
| | ATCTCTAGCCTGCAGCCTGAGGATTTCGCCACATACTATTGC |
| | CAGCAGTCCTATTCTACACCACTGACCTTTGGCGGCGGCAC |
| | CAAGGTGGAGATCAAGACCACAACCCCAGCACCCAGACCC |
| | CCTACACCTGCACCAACCATCGCATCCCAGCCACTGTCTCTG |
| | CGGCCCGAGGCATGTAGGCCAGCAGCAGGAGGAGCAGTGC |
| | ACACCAGGGGCCTGGACTTCGCCTGCGATATCTACATTTGG |
| | GCACCACTGGCAGGAACCTGTGGCGTGCTGCTCCTGAGCCT |
| | GGTCATCACCCTGTACTGCAAGCGCGGCCGGAAGAAGCTGC |
| | TGTATATCTTCAAGCAGCCCTTCATGCGGCCCGTGCAGACA |
| | ACCCAGGAGGAGGACGGCTGCTCCTGTCGGTTCCCAGAAGA |
| | GGAGGAGGGAGGATGTGAGCTGAGGGTGAAGTTTAGCCGG |
| | TCCGCCGATGCACCAGCATACCAGCAGGGCCAGAATCAGCT |
| | GTATAACGAGCTGAATCTGGGCCGGAGAGAGGAGTACGAC |
| | GTGCTGGATAAGAGGAGGGGAAGGGATCCTGAGATGGGAG |
| | GCAAGCCCCGGAGAAAGAACCCTCAGGAGGGCCTGTACAA |
| | TGAGCTGCAGAAGGACAAGATGGCCGAGGCCTATTCCGAG |
| | ATCGGCATGAAGGGAGAGAGGCGCCGGGGCAAGGGACACG |
| | ATGGCCTGTACCAGGGCCTGTCTACAGCCACCAAGGACACC |
| | TATGATGCCCTGCATATGCAGGCACTGCCTCCAAGGTGA |
| | (SEQ ID NO: 8) |
| | Amino acid: |
| | MALPVTALLLPLALLLHAARPQVQLQQSGPGLVEPSQTLSLTC |
| | AISGDSVSSNSAAWNWIRQSPSRGLEWLGRTYYRSTWYNDYA |
| | GSVKSRITINPDTSKNQFSLQLTSVTPEDTAVYYCTRSRHNTFR |
| | GMDVWGQGTLVTVSGGGGSGGGGSGGGGSDIQLTQSPSSLSA |
| | SVGDRVTITCRASQSISSYLNWYQQKPGKAPKLLIYAASSLQSG |
| | VPSRFSGSGSGTDFTLTISSLQPEDFATYYCQQSYSTPLTFGGGT |
| | KVEIKTTTPAPRPPTPAPTIASQPLSLRPEACRPAAGGAVHTRG |
| | LDFACDIYIWAPLAGTCGVLLLSLVITLYCKRGRKKLLYIFKQP |
| | FMRPVQTTQEEDGCSCRFPEEEEGGCELRVKFSRSADAPAYQQ |
| | GQNQLYNELNLGRREEYDVLDKRRGRDPEMGGKPRRKNPQE |
| | GLYNELQKDKMAEAYSEIGMKGERRRGKGHDGLYQGLSTAT |
| | KDTYDALHMQALPPR |
| | (SEQ ID NO: 9) |
| pCLS29967; B-E12 | Nucleic acid: |
| | ATGGCTCTGCCCGTCACCGCTCTGCTGCTGCCACTGGCCCTG |
| | CTGCTGCACGCAGCCAGACCCCAGGTGCAGCTGCAGCAGAG |
| | CGGCCCTGGCCTGGTGCAGCCATCCCAGACACTGTCTCTGA |
| | CCTGCGTGATCAGCGGCGACTCCGTGAGCTCCAACTCTGCC |
| | ACATGGAATTGGATCAGACAGAGCCCATCCAGGGGCCTGG |
| | AGTGGCTGGGACGCACCTACTATCGGAGCAAGTGGTACAAC |
| | GACTATGCCGTGTCTGTGAAGAGCAGAATCACAATCAACCC |
| | CGATACCTCTAAGAATCAGTTCAGCCTGCAGCTGAATTCCG |
| | TGACACCTGAGGATACCGCCGTGTACTATTGCGCCAGGGAC |
| | GGCGATGGAGGAAGCTACTATGACTACTATTACTATGGCAT |
| | GGACGTGTGGGGCCAGGGCACCACAGTGACAGTGTCTGGA |
| | GGAGGAGGAAGCGGAGGAGGAGGATCCGGCGGCGGCGGCT |
| | CTGACATCCAGCTGACACAGTCCCCTTCTAGCCTGTCTACCA |
| | GCGTGGGCGATCGCGTGACAATCACCTGTCGGGCCTCCCAG |
| | TCTATCAGCACCTACCTGAACTGGTATCAGCAGAAGCCCGG |
| | CAAGGCCCCTAAGCTGCTGATCTACGCAGCAAGCAATCTGC |
| | AGTCCGGAGTGCCATCTCGCTTCTCCGGCTCTGGCAGCGGC |
| | ACAGACTTTACACTGACCATCTCCTCTCTGCAGCCTGAGGAT |
| | TTCGCCACCTACTTTTGCCAGCAGTCCTATACCACACCAATC |
| | ACATTCGGCCAGGGCACCAGACTGGAGATCAAGACCACAA |
| | CCCCAGCACCCAGGCCCCCTACACCTGCACCAACCATCGCA |
| | AGCCAGCCACTGTCCCTGCGCCCTGAGGCATGTAGGCCAGC |
| | AGCAGGAGGAGCAGTGCACACCAGAGGCCTGGACTTTGCCT |
| | GCGATATTTACATCTGGGCACCACTGGCAGGAACATGTGGC |
| | GTGCTGCTCCTGAGCCTGGTCATCACCCTGTACTGCAAGAG |
| | AGGCAGGAAGAAGCTGCTGTATATCTTCAAGCAGCCCTTCA |
| | TGCGGCCCGTGCAGACAACCCAGGAGGAGGACGGCTGCTCT |
| | TGTCGGTTCCCAGAAGAGGAGGAGGGCGGCTGTGAGCTGA |
| | GAGTGAAGTTTTCCAGGTCTGCCGATGCACCAGCATACCAG |
| | CAGGGACAGAACCAGCTGTATAACGAGCTGAATCTGGGCC |
| | GGAGAGAGGAGTACGACGTGCTGGATAAGAGGAGGGGACG |
| | GGACCCTGAGATGGGAGGCAAGCCCCGGAGAAAGAACCCT |
| | CAGGAGGGCCTGTACAATGAGCTGCAGAAGGACAAGATGG |
| | CCGAGGCCTATAGCGAGATCGGCATGAAGGGAGAGAGGCG |
| | CCGGGGCAAGGGACACGATGGCCTGTACCAGGGCCTGTCCA |
| | CAGCCACCAAGGACACCTATGATGCCCTGCATATGCAGGCA |
| | CTGCCTCCAAGGTGA |
| | (SEQ ID NO: 10) |
| | Amino acid: |
| | MALPVTALLLPLALLLHAARPQVQLQQSGPGLVQPSQTLSLTC |
| | VISGDSVSSNSATWNWIRQSPSRGLEWLGRTYYRSKWYNDYA |
| | VSVKSRITINPDTSKNQFSLQLNSVTPEDTAVYYCARDGDGGS |
| | YYDYYYYGMDVWGQGTTVTVSGGGGSGGGGSGGGGSDIQL |
| | TQSPSSLSTSVGDRVTITCRASQSISTYLNWYQQKPGKAPKLLI |
| | YAASNLQSGVPSRFSGSGSGTDFTLTISSLQPEDFATYFCQQSY |
| | TTPITFGQGTRLEIKTTTPAPRPPTPAPTIASQPLSLRPEACRPAA |
| | GGAVHTRGLDFACDIYIWAPLAGTCGVLLLSLVITLYCKRGRK |
| | KLLYIFKQPFMRPVQTTQEEDGCSCRFPEEEEGGCELRVKFSRS |
| | ADAPAYQQGQNQLYNELNLGRREEYDVLDKRRGRDPEMGGK |
| | PRRKNPQEGLYNELQKDKMAEAYSEIGMKGERRRGKGHDGL |
| | YQGLSTATKDTYDALHMQALPPR |
| | (SEQ ID NO: 11) |
| pCLS29970; A-B2 | Nucleic acid: |
| | ATGGCTCTGCCCGTCACCGCTCTGCTGCTGCCACTGGCCCTG |
| | CTGCTGCACGCAGCCAGACCCCAGGTGCAGCTGCAGCAGTC |
| | CGGCCCTGGCCTGGTGAAGCCATCTCAGACACTGAGCCTGA |
| | CCTGCGCCATCTCCGGCGACTCTGTGAGCTCCAACTCCGCC |
| | GCCTGGAATTGGATCAGACAGAGCCCATCCAGGGGCCTGGA |
| | GTGGCTGGGACGCACCTACTATCGGAGCGCCTGGTACAACG |
| | ACTATGCCGTGAGCGTGAAGTCCAGAATCACAATCAACCCC |
| | GATACCTCTAAGAATCAGTTCAGCCTGCAGCTGTCTAGCGT |
| | GACACCTGAGGATACCGCCGTGTACTATTGCGCCAGGGACG |
| | TGGAGGGCTTTGATTACTGGGGCCAGGGCACACTGGTGACC |
| | GTGTCCGGCGGCGGCGGCTCTGGAGGAGGAGGAAGCGGAG |
| | GAGGAGGATCCGACATCGTGATGACACAGACCCCTTCCTCT |
| | CTGTCTGCCAGCGTGGGCGATCGCGTGACAATCACCTGTCG |
| | GGCCTCCCAGTCTATCAGCTCCTACCTGAATTGGTATCAGCA |
| | GAAGCCCGGCAAGGCCCCTAAGCTGCTGATCTACGCAGCAT |
| | CTAGCCTGCAGTCCGGAGTGCCATCTCGCTTCAGCGGATCC |
| | GGCTCTGGCACAGACTTTACACTGACCATCTCCTCTCTGCAG |
| | CCTGAGGATTTCGCCACCTACTATTGCCAGCAGAGCTATTCC |
| | ACACCAATCACCTTTGGCCAGGGCACAAGACTGGAGATCAA |
| | GACCACAACCCCAGCACCCAGGCCCCCTACACCTGCACCAA |
| | CCATCGCAAGCCAGCCACTGTCCCTGCGCCCTGAGGCATGT |
| | AGGCCAGCAGCAGGAGGAGCAGTGCACACCAGAGGCCTGG |
| | ACTTCGCCTGCGATATTTACATCTGGGCACCACTGGCAGGA |
| | ACATGTGGCGTGCTGCTCCTGAGCCTGGTCATCACCCTGTAC |
| | TGCAAGAGAGGCAGGAAGAAGCTGCTGTATATCTTCAAGCA |
| | GCCCTTCATGCGGCCCGTGCAGACAACCCAGGAGGAGGAC |
| | GGCTGCAGCTGTCGGTTCCCAGAAGAGGAGGAGGGCGGCT |
| | GTGAGCTGAGAGTGAAGTTTTCTAGGAGCGCCGATGCACCA |
| | GCATACCAGCAGGGACAGAACCAGCTGTATAACGAGCTGA |
| | ATCTGGGCCGGAGAGAGGAGTACGACGTGCTGGATAAGAG |
| | GAGGGGACGGGACCCTGAGATGGGAGGCAAGCCCCGGAGA |
| | AAGAACCCTCAGGAGGGCCTGTACAATGAGCTGCAGAAGG |
| | ACAAGATGGCCGAGGCCTATTCTGAGATCGGCATGAAGGG |
| | AGAGAGGCGCCGGGGCAAGGGACACGATGGCCTGTACCAG |
| | GGCCTGAGCACAGCCACCAAGGACACCTATGATGCCCTGCA |
| | TATGCAGGCACTGCCTCCAAGGTGA |
| | (SEQ ID NO: 12) |
| | Amino acid: |
| | MALPVTALLLPLALLLHAARPQVQLQQSGPGLVKPSQTLSLTC |
| | AISGDSVSSNSAAWNWIRQSPSRGLEWLGRTYYRSAWYNDYA |
| | VSVKSRITINPDTSKNQFSLQLSSVTPEDTAVYYCARDVEGFDY |
| | WGQGTLVTVSGGGGSGGGGSGGGGSDIVMTQTPSSLSASVGD |
| | RVTITCRASQSISSYLNWYQQKPGKAPKLLIYAASSLQSGVPSR |
| | FSGSGSGTDFTLTISSLQPEDFATYYCQQSYSTPITFGQGTRLEI |
| | KTTTPAPRPPTPAPTIASQPLSLRPEACRPAAGGAVHTRGLDFA |
| | CDIYIWAPLAGTCGVLLLSLVITLYCKRGRKKLLYIFKQPFMRP |
| | VQTTQEEDGCSCRFPEEEEGGCELRVKFSRSADAPAYQQGQN |
| | QLYNELNLGRREEYDVLDKRRGRDPEMGGKPRRKNPQEGLY |
| | NELQKDKMAEAYSEIGMKGERRRGKGHDGLYQGLSTATKDT |
| | YDALHMQALPPR |
| | (SEQ ID NO: 13) |
| pCLS29971; A-D4 | Nucleic acid: |
| | ATGGCTCTGCCCGTCACCGCTCTGCTGCTGCCTCTGGCCCTG |
| | CTGCTGCACGCAGCCCGCCCACAGGTGCAGCTGCAGCAGAG |
| | CGGCCCCGGCCTGGTGAAGCCTAGCCAGACACTGTCCCTGA |
| | CCTGCGCAATCTCCGGCGACAGCGTGTCCGGAAACAGGGCC |
| | ACATGGAATTGGATCAGACAGTCTCCAAGCAGGGGCCTGGA |
| | GTGGCTGGGAAGGACCTACTATCGGTCCGCCTGGTACAACG |
| | ACTATGCCGTGTCTGTGAAGGGCCGCATCACATTCAACCCA |
| | GATACCAGCAAGAATCAGTTTTCCCTGCAGCTGAATTCTGT |
| | GACACCCGAGGATACCGCCGTGTACTATTGCGCCAGAGGCG |
| | AGAGCGGAGCAGCAGCAGACGCCTTCGATATCTGGGGCCA |
| | GGGCACCACAGTGACAGTGAGCGGAGGAGGAGGATCCGGC |
| | GGAGGAGGCTCTGGCGGCGGCGGCAGCGACATCCAGCTGA |
| | CCCAGAGCCCACCTTCCCTGTCTGCCAGCGTGGGCGATCGC |
| | GTGACAATCACCTGTCGGGCCTCCCAGTCTATCAGCTCCTAC |
| | CTGAACTGGTATCAGCAGAAGCCAGGCAAGGCCCCCAAGCT |
| | GCTGATCTACGCAGCATCTAGCCTGCAGTCTGGAGTGCCAA |
| | GCAGATTCAGCGGATCCGGATTCGGCACAGACTTTACACTG |
| | ACCATCTCCTCTCTGCAGCCCGAGGATTTCGCCACCTACTAT |
| | TGCCAGCAGTCTTATAGCACACCTCAGACCTTTGGCCAGGG |
| | CACCAAGGTGGACATCAAGACCACAACCCCTGCACCAAGA |
| | CCACCAACACCAGCACCTACCATCGCATCCCAGCCACTGTC |
| | TCTGCGCCCCGAGGCATGTAGGCCTGCAGCAGGCGGCGCCG |
| | TGCACACCAGGGGCCTGGACTTTGCCTGCGATATTTACATCT |
| | GGGCACCTCTGGCAGGAACATGTGGCGTGCTGCTCCTGAGC |
| | CTGGTCATCACCCTGTACTGCAAGAGAGGCAGGAAGAAGCT |
| | GCTGTATATCTTCAAGCAGCCCTTCATGCGGCCCGTGCAGA |
| | CAACCCAGGAGGAGGACGGCTGCTCCTGTAGGTTCCCTGAA |
| | GAGGAGGAGGGCGGCTGTGAGCTGAGAGTGAAGTTTTCCA |
| | GGTCTGCCGATGCACCAGCATACCAGCAGGGACAGAATCA |
| | GCTGTATAACGAGCTGAATCTGGGCCGGAGAGAGGAGTAC |
| | GACGTGCTGGATAAGAGGAGGGGACGGGATCCCGAGATGG |
| | GAGGCAAGCCACGGAGAAAGAACCCCCAGGAGGGCCTGTA |
| | CAATGAGCTGCAGAAGGACAAGATGGCCGAGGCCTATTCTG |
| | AGATCGGCATGAAGGGAGAGAGGCGCCGGGGCAAGGGACA |
| | CGATGGCCTGTACCAGGGCCTGTCCACAGCCACCAAGGACA |
| | CCTATGATGCCCTGCATATGCAGGCACTGCCTCCAAGGTGA |
| | (SEQ ID NO: 14) |
| | Amino acid: |
| | MALPVTALLLPLALLLHAARPQVQLQQSGPGLVKPSQTLSLTC |
| | AISGDSVSGNRATWNWIRQSPSRGLEWLGRTYYRSAWYNDY |
| | AVSVKGRITFNPDTSKNQFSLQLNSVTPEDTAVYYCARGESGA |
| | AADAFDIWGQGTTVTVSGGGGSGGGGSGGGGSDIQLTQSPPSL |
| | SASVGDRVTITCRASQSISSYLNWYQQKPGKAPKLLIYAASSLQ |
| | SGVPSRFSGSGFGTDFTLTISSLQPEDFATYYCQQSYSTPQTFGQ |
| | GTKVDIKTTTPAPRPPTPAPTIASQPLSLRPEACRPAAGGAVHT |
| | RGLDFACDIYIWAPLAGTCGVLLLSLVITLYCKRGRKKLLYIFK |
| | QPFMRPVQTTQEEDGCSCRFPEEEEGGCELRVKFSRSADAPAY |
| | QQGQNQLYNELNLGRREEYDVLDKRRGRDPEMGGKPRRKNP |
| | QEGLYNELQKDKMAEAYSEIGMKGERRRGKGHDGLYQGLST |
| | ATKDTYDALHMQALPPR |
| | (SEQ ID NO: 15) |
| pCLS28154; m971 | Nucleic acid: |
| | ATGGCTCTGCCCGTCACCGCTCTGCTGCTGCCACTGGCCCTG |
| | CTGCTGCACGCAGCAAGACCACAGGTGCAGCTGCAGCAGA |
| | GCGGCCCTGGCCTGGTGAAGCCAAGCCAGACACTGTCCCTG |
| | ACCTGCGCCATCAGCGGCGATTCCGTGAGCTCCAACTCCGC |
| | CGCCTGGAATTGGATCAGGCAGTCCCCTTCTCGGGGCCTGG |
| | AGTGGCTGGGAAGGACATACTATCGGTCTAAGTGGTACAAC |
| | GATTATGCCGTGTCTGTGAAGAGCAGAATCACAATCAACCC |
| | TGACACCTCCAAGAATCAGTTCTCTCTGCAGCTGAATAGCG |
| | TGACACCAGAGGACACCGCCGTGTACTATTGCGCCAGGGAG |
| | GTGACCGGCGACCTGGAGGATGCCTTTGACATCTGGGGCCA |
| | GGGCACAATGGTGACCGTGTCTAGCGGAGGAGGAGGATCC |
| | GGAGGAGGAGGATCTGGCGGCGGCGGCAGCGATATCCAGA |
| | TGACACAGTCCCCATCCTCTCTGAGCGCCTCCGTGGGCGAC |
| | AGAGTGACAATCACCTGTAGGGCCTCCCAGACCATCTGGTC |
| | TTACCTGAACTGGTATCAGCAGAGGCCCGGCAAGGCCCCTA |
| | ATCTGCTGATCTACGCAGCAAGCTCCCTGCAGAGCGGAGTG |
| | CCATCCAGATTCTCTGGCAGGGGCTCCGGCACAGACTTCAC |
| | CCTGACCATCTCTAGCCTGCAGGCCGAGGACTTCGCCACCT |
| | ACTATTGCCAGCAGTCTTATAGCATCCCCCAGACATTTGGCC |
| | AGGGCACCAAGCTGGAGATCAAGACCACAACCCCAGCACC |
| | AAGGCCACCTACACCTGCACCAACCATCGCCTCTCAGCCCC |
| | TGAGCCTGAGACCTGAGGCATGTAGGCCAGCAGCAGGAGG |
| | AGCAGTCCATACAAGGGGTCTGGATTTTGCATGCGACATCT |
| | ACATCTGGGCACCTCTGGCAGGAACATGTGGCGTGCTCCTG |
| | CTCAGCCTGGTCATCACCCTGTACTGCAAGAGAGGCAGGAA |
| | GAAGCTGCTGTATATCTTCAAGCAGCCCTTCATGCGCCCCGT |
| | GCAGACAACCCAGGAGGAGGATGGCTGCTCCTGTAGGTTCC |
| | CAGAAGAGGAGGAGGGAGGATGTGAGCTGCGCGTGAAGTT |
| | TTCCCGGTCTGCCGACGCACCTGCATACCAGCAGGGCCAGA |
| | ACCAGCTGTATAACGAGCTGAATCTGGGCCGGAGAGAGGA |
| | GTACGATGTGCTGGACAAGAGGCGCGGCAGAGATCCAGAG |
| | ATGGGCGGCAAGCCCCGGAGAAAGAACCCTCAGGAGGGCC |
| | TGTACAATGAGCTGCAGAAGGATAAGATGGCCGAGGCCTAT |
| | TCTGAGATCGGCATGAAGGGAGAGAGGCGCCGGGGCAAGG |
| | GACACGACGGACTGTACCAGGGACTGAGCACAGCCACCAA |
| | GGATACCTATGACGCCCTGCATATGCAGGCACTGCCTCCAA |
| | GGTGA |
| | (SEQ ID NO: 16) |
| | Amino acid: |
| | MALPVTALLLPLALLLHAARPQVQLQQSGPGLVKPSQTLSLTC |
| | AISGDSVSSNSAAWNWIRQSPSRGLEWLGRTYYRSKWYNDYA |
| | VSVKSRITINPDTSKNQFSLQLNSVTPEDTAVYYCAREVTGDLE |
| | DAFDIWGQGTMVTVSSGGGGSGGGGSGGGGSDIQMTQSPSSL |
| | SASVGDRVTITCRASQTIWSYLNWYQQRPGKAPNLLIYAASSL |
| | QSGVPSRFSGRGSGTDFTLTISSLQAEDFATYYCQQSYSIPQTFG |
| | QGTKLEIKTTTPAPRPPTPAPTIASQPLSLRPEACRPAAGGAVHT |
| | RGLDFACDIYIWAPLAGTCGVLLLSLVITLYCKRGRKKLLYIFK |
| | QPFMRPVQTTQEEDGCSCRFPEEEEGGCELRVKFSRSADAPAY |
| | QQGQNQLYNELNLGRREEYDVLDKRRGRDPEMGGKPRRKNP |
| | QEGLYNELQKDKMAEAYSEIGMKGERRRGKGHDGLYQGLST |
| | ATKDTYDALHMQALPPR |
| | (SEQ ID NO: 17) |

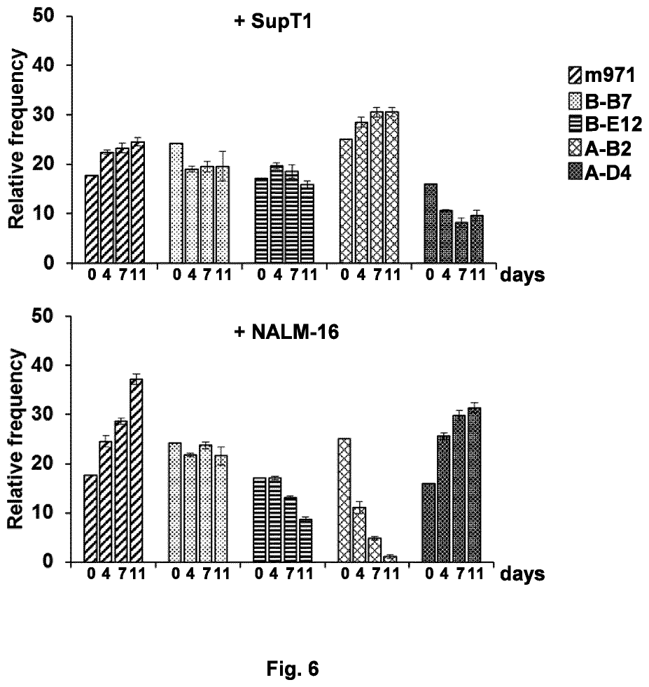

Sequencing by Illumina MiSeq gave an average of 61,138 analyzable reads (standard deviation of 9,753). These reads were aligned against the sequences of the different scFv and a score of alignment was calculated. The scFv selected corresponds to the one with the highest score. The results obtained are summarized in FIG. 6. The results clearly demonstrate that there is an important enrichment of both CARs m971 and A-D4 over time. B-B7 CAR appears to be stable overtime while the relative frequency of A-B2 CAR slowly decreases after two reactivations. The relative frequency of B-E12 CAR is strikingly reduced very early after only one reactivation. Since those frequencies are relative frequencies, it is difficult to determine whether the mechanism observed is an enrichment of some CARs or a loss of others. Nevertheless, the selection of the best candidates is very simple with this approach, the two best candidates being CARs m971 and A-D4. B-B7 CAR is still a very good option since its relative frequency is stable overtime. A-B2 and B-E12 are poor candidates as their relative frequencies rapidly dropped down as early as after one reactivation or two.

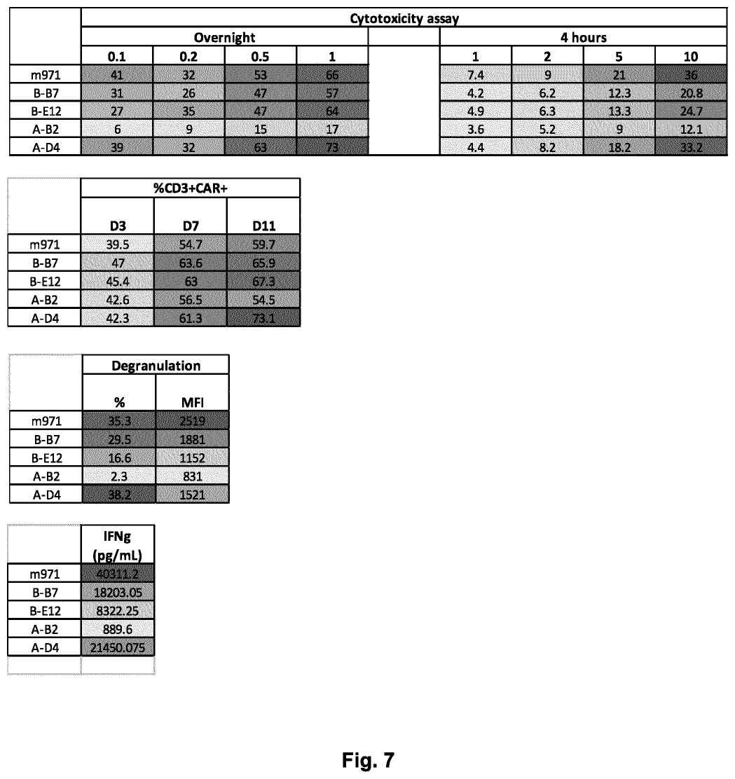

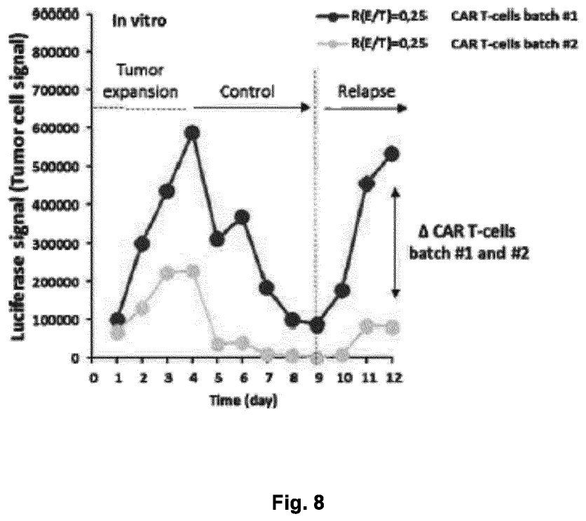

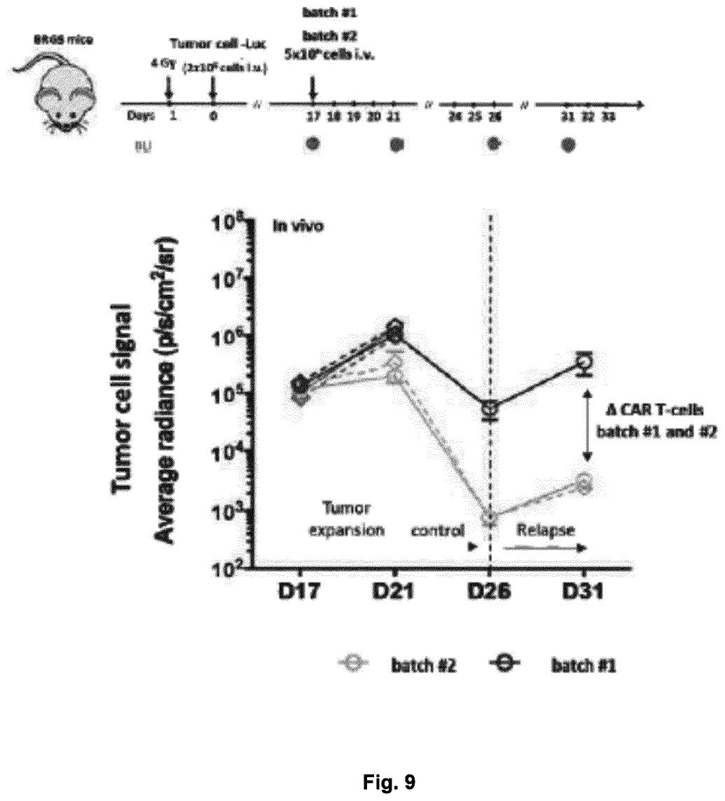

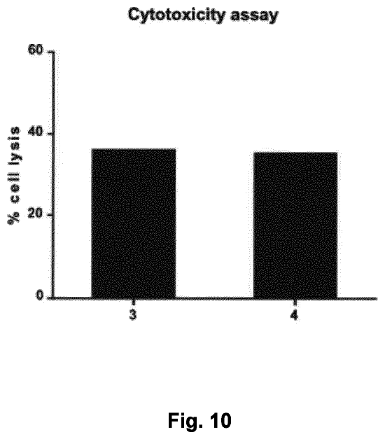

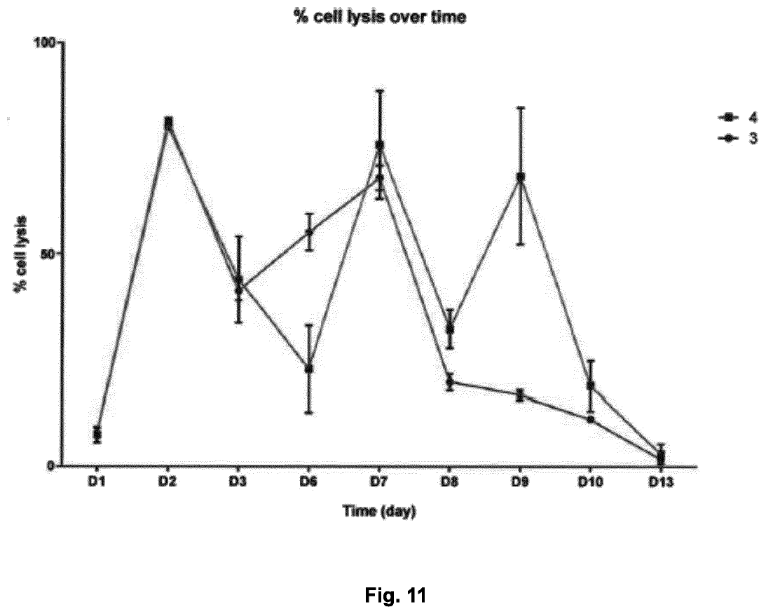

To validate the hypothesis that the best CAR candidates can be identified using an in vitro strategy based on deep sequencing analysis, the in vitro functional activities of the chosen CARs had been previously determined by in vitro assays (cytotoxicity assay, degranulation assay and IFN-γ measurement) and are summarized in FIG. 7. The results obtained in this first screen highlighted that the 3 best candidates were m971, A-D4 and B-B7 CARs. It was impossible to predict which of either A-D4 or B-B7 would be the CAR which would have the strongest activity. Using this new strategy, it seems that B-B7 candidate will not be as good as A-D4 since its relative frequency among the pool of CARs selected does not change along the course of the experiment. B-E12 was also considered a good candidate even though its degranulation activity and IFN-γ secretion capability seemed to be a bit lower as compared to the other good candidates. Using this strategy, it appears that there is a loss of B-E12 after many reactivations which might suggest that this CAR is not such a good candidate. The results obtained using this new strategy confirmed the results obtained from the preliminary screen and demonstrated that A-B2 CAR is for sure not a good CD22 candidate.

### Example 2. In Vitro Selection of CLL1 CART Cells

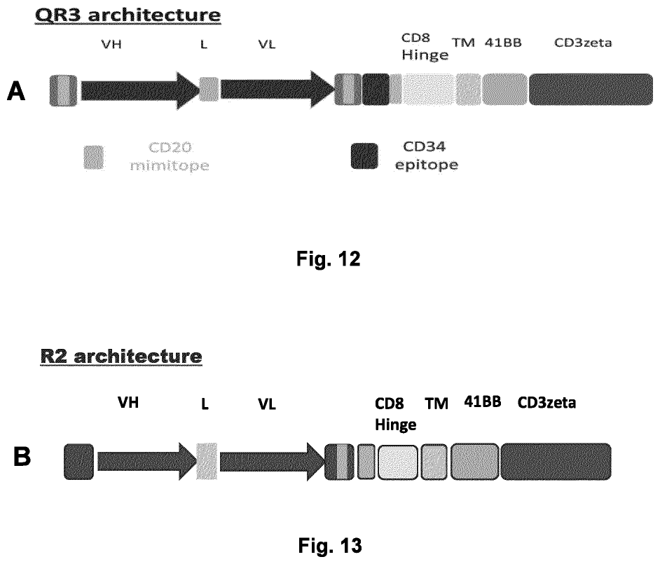

Anti-CLL1 chimeric antigen receptors harboring “QR3” architecture as disclosed in FIG. 12 were produced and tested in different cell lines to individually assess their efficiencies against various CLL1 positive cancer cell lines.

Then, the approach of pooling equimolar populations of T-cells endowed with the different CARs was pursued according to the invention to compare their activation over time and determine which are the most competitive.

### 2.1 CAR Sequences and Architectures

Twelve sequences already described in the literature were analyzed and the variable regions matching with the germline coding sequences were selected to be designed in the QR3 architecture (FIG. 13) harboring a CD8 hinge and cloned in pSew-BFP backbone or pCCL. The corresponding lentiviral vectors of these CARs were produced and titrated with Jurkat cells based on BFP expression, while those generated on pCCL backbone could only by semi-quantified using p24 ELISA.

TABLE 2
Affinity of different antibodies for CLL-1.

| Ab | Kd (nM) |
|---|---|
| M2 | 0.205 |
| M29 | 1.48 |
| M31 | 0.611 |
| M26 | 0.214 |
| 21.26 | 15.3 |
| M5 | 0.553 |
| G4 | 0.053 |
| 1075.7 | 0.36 |
| G12 | 0.07 |
| M22 | 0.388 |
| Sc02-161 | |
| Sc02-357 | |

TABLE 3
polynucleotide sequences related to anti CLL1 CARs used in
Example 2

| Name of the sequence | Nucleic Acid/Amino Acid Sequence |
|---|---|
| Oligo CLL1-1-F | AAGACTCGGCAGCATCTCCATGGCTCCAGCAGAAGCC |
| | (SEQ ID NO: 18) |
| Oligo CLL1-1-R_ | GCGATCGTCACTGTTCTCCAGGAGGCAGTTAGTAATC |
| | GGCG |
| | (SEQ ID NO: 19) |
| CAR M2-QR3 | GCTCTGCCCGTCACCGCTCTGCTGCTGCCACTGGCCCTG |
| | CTGCTGCACGCCGCCAGACCCGGAGGAGGAGGAAGCT |
| | GCCCCTATTCAAACCCATCACTGTGCTCAGGAGGAGGA |
| | GGATCAGGAGGAGGAGGAAGCGAAGTCCAGCTGCGGC |
| | AGAGCGGCCCCGAGCTGGTGAAGCCTGGCGCCTCCGTG |
| | AAGATGTCTTGCAAGGCCAGCGGCTACACCTTCACATC |
| | CTACTTCATGCACTGGGTGAAGCAGAAGCCTGGCCAGG |
| | GCCTGGAGTGGATCGGCTTCATCAACCCATACAATGAC |
| | GGCACCAAGTATAACGAGAAGTTTAAGGGCAAGGCCA |
| | CCCTGACAAGCGATAAGAGCAGCAGCACCGCCTACATG |
| | GAGCTGAATAGCCTGACATCCGAGGACTCTGCCGTGTA |
| | CTATTGCACAAGGGACGATGGCTACTATGACTACGCTA |
| | TGGACTATTGGGGCCAGGGCACCTCCGTGACCGTGAGC |
| | AGCGGCGGAGGCGGCTCTGGCGGAGGAGGCAGCGGCG |
| | GAGGAGGCTCCGACATCCAGATGACCCAGAGCCCTTCT |
| | AGCCTGAGCGCCTCCCTGGGAGAGAGGGTGTCCCTGAC |
| | ATGTCGCGCCTCCCAGGAGATCAGCGTGTACCTGAGCT |
| | GGCTCCAGCAGAAGCCCGACGGCACCATCAAGCGCCTG |
| | ATCTATGCCGCCTCCACACTGGATTCTGGCGTGCCTGAG |
| | CGGTTCTCTGGCAGCAGATCCGGCTCTGACTACTCCCTG |
| | ACCATCTCCTCTCTGGAGTCTGAGGACTTCGCCGATTAC |
| | TACTGCCTCCAGTATGCCAGCTACCCATATACCTTTGGC |
| | GGCGGCACAAAGCTGGAGATCAAGGGCTCTGGCGGCG |
| | GCGGCAGCTGCCCATACAGCAACCCCAGCCTGTGCAGC |
| | GGCGGCGGGGGCTCTGAGCTGCCCACCCAGGGCACCTT |
| | CAGCAACGTGTCCACCAACGTGAGCCCAGCAAAGCCTA |
| | CCACAACCGCATGCCCTTATTCTAATCCCAGCCTGTGCA |
| | CAACCACACCAGCACCCAGACCCCCTACCCCTGCACCA |
| | ACAATCGCCAGCCAGCCACTGAGCCTGCGGCCCGAGGC |
| | ATGTAGACCCGCCGCTGGAGGAGCCGTGCATACTAGAG |
| | GACTGGACTTCGCATGTGACATCTATATCTGGGCACCA |
| | CTGGCCGGAACATGTGGCGTGCTGCTGCTGTCACTGGT |
| | CATTACACTGTACTGTAAGCGAGGCCGGAAGAAACTGC |
| | TGTATATTTTCAAACAGCCCTTTATGAGACCTGTGCAGA |
| | CTACCCAGGAGGAAGACGGCTGCAGCTGTAGGTTCCCC |
| | GAGGAAGAGGAAGGCGGGTGTGAGCTGAGGGTCAAGT |
| | TTAGCCGCTCCGCAGATGCCCCTGCTTACCAGCAGGGG |
| | CAGAATCAGCTGTATAACGAGCTGAATCTGGGACGGAG |
| | AGAGGAATACGACGTGCTGGATAAAAGGCGCGGGAGA |
| | GACCCCGAAATGGGAGGCAAGCCACGACGGAAAAACC |
| | CCCAGGAGGGCCTGTACAATGAACTGCAGAAGGACAA |
| | AATGGCAGAGGCCTATAGTGAAATCGGGATGAAGGGA |
| | GAGAGAAGGCGCGGCAAAGGGCACGATGGCCTGTACC |
| | AGGGGCTGTCTACTGCCACCAAGGACACCTATGATGCT |
| | CTGCATATGCAGGCACTGCCTCCAAGGTGA |
| | (SEQ ID NO: 20) |
| CAR M26-QR3 | GCTCTGCCCGTCACCGCTCTGCTGCTGCCACTGGCCCTG |
| | CTGCTGCACGCCGCCAGACCCGGAGGAGGAGGCTCATG |
| | CCCTTATTCCAACCCTTCACTGTGCTCAGGAGGAGGAG |
| | GATCAGGCGGAGGAGGCTCAGAGGTCCAGCTGCAACA |
| | GAGCGGCCCCGAGCTGGTGAAGCCTGGCGCCAGCGTGA |
| | AGATGTCCTGCAAGGCCAGCGGCTACACCTTCACATCC |
| | TACTTCATCCACTGGGTGAAGCAGAAGCCTGGCCAGGG |
| | CCTGGAGTGGATCGGCTTCATCAACCCATACAATGACG |
| | GCAGCAAGTATAACGAGAAGTTTAAGGGCAAGGCCAC |
| | CCTGACATCCGATAAGAGCAGCAGCACCGCCTACATGG |
| | AGCTGAGCAGCCTGACCAGCGAGGACTCCGCCGTGTAC |
| | TATTGCACCAGGGACGATGGCTACTATGGCTACGCTAT |
| | GGACTATTGGGGCCAGGGCACCAGCGTGACAGTGTCTA |
| | GCGGCGGAGGAGGCAGCGGCGGAGGAGGCTCCGGCGG |
| | CGGCGGCTCTGACATCCAGATGACACAGTCCCCTTCCTC |
| | TCTGTCTGCCAGCCTGGGCGAGAGGGTGTCTCTGACCT |
| | GTCGCGCCACACAGGAGCTGAGCGGCTACCTGTCCTGG |
| | CTCCAGCAGAAGCCCGACGGCACCATCAAGAGACTGAT |
| | CTATGCCGCCTCTACACTGGATAGCGGCGTGCCTAAGC |
| | GGTTCAGCGGCAATAGATCCGGCTCTGATTACTCTCTGA |
| | CCATCAGCTCCCTGGAGAGCGAGGACTTCGCCGATTAC |
| | TACTGCCTCCAGTATGCCATCTACCCATATACCTTTGGC |
| | GGCGGCACAAAGCTGGAGATCAAGGGCAGCGGCGGCG |
| | GCGGCTCCTGCCCATACTCTAACCCCAGCCTGTGCAGC |
| | GGCGGCGGGGGCTCTGAGCTGCCCACCCAGGGCACATT |
| | TTCTAACGTGAGCACCAACGTGAGCCCAGCAAAGCCTA |
| | CCACAACCGCATGCCCTTATTCCAATCCCAGCCTGTGCA |
| | CAACCACACCAGCACCCAGACCCCCTACCCCTGCACCA |
| | ACAATCGCCTCCCAGCCACTGAGCCTGCGGCCCGAGGC |
| | ATGTAGACCCGCCGCCGGAGGCGCTGTGCATACCCGAG |
| | GACTGGACTTTGCTTGCGACATCTATATCTGGGCACCAC |
| | TGGCCGGAACATGTGGCGTGCTGCTGCTGTCACTGGTC |
| | ATTACACTGTACTGTAAGCGAGGCCGGAAGAAACTGCT |
| | GTATATTTTCAAACAGCCCTTTATGAGACCTGTGCAGAC |
| | TACCCAGGAGGAAGACGGCTGCAGCTGTAGGTTCCCCG |
| | AGGAAGAGGAAGGCGGGTGTGAGCTGAGGGTCAAGTT |
| | TAGCCGCTCCGCAGATGCCCCTGCTTACCAGCAGGGGC |
| | AGAATCAGCTGTATAACGAGCTGAATCTGGGACGGAGA |
| | GAGGAATACGACGTGCTGGATAAAAGGCGCGGGAGAG |
| | ACCCCGAAATGGGAGGCAAGCCACGACGGAAAAACCC |
| | CCAGGAGGGCCTGTACAATGAACTGCAGAAGGACAAA |
| | ATGGCAGAGGCCTATAGTGAAATCGGGATGAAGGGAG |
| | AGAGAAGGCGCGGCAAAGGGCACGATGGCCTGTACCA |
| | GGGGCTGTCTACTGCCACCAAGGACACCTATGATGCTC |
| | TGCATATGCAGGCACTGCCTCCAAGGTGA |
| | (SEQ ID NO: 21) |
| CAR M2-R2 | GCTCTGCCCGTCACCGCTCTGCTGCTGCCACTGGCCCTG |
| | CTGCTGCACGCCGCCAGACCCGAAGTCCAGCTGAGACA |
| | GTCAGGACCCGAACTGGTGAAACCCGGCGCAAGTGTGA |
| | AGATGAGTTGTAAAGCCTCAGGATATACATTCACATCC |
| | TACTTCATGCACTGGGTGAAGCAGAAGCCTGGCCAGGG |
| | CCTGGAGTGGATCGGCTTCATCAACCCTTACAATGACG |
| | GCACCAAGTATAACGAGAAGTTTAAGGGCAAGGCCAC |
| | ACTGACCAGCGATAAGAGCAGCAGCACCGCCTACATGG |
| | AGCTGAATAGCCTGACCTCCGAGGACTCTGCCGTGTAC |
| | TATTGCACAAGGGACGATGGCTACTATGACTACGCTAT |
| | GGACTATTGGGGCCAGGGCACATCCGTGACCGTGAGCA |
| | GCGGCGGAGGAGGCAGCGGCGGAGGAGGCTCCGGCGG |
| | CGGCGGCTCTGACATCCAGATGACACAGTCTCCCTCTA |
| | GCCTGAGCGCCTCCCTGGGAGAGAGGGTGAGCCTGACC |
| | TGTCGCGCCAGCCAGGAGATCAGCGTGTACCTGTCTTG |
| | GCTCCAGCAGAAGCCTGACGGCACAATCAAGAGACTGA |
| | TCTATGCAGCCAGCACCCTGGATTCCGGCGTGCCAGAG |
| | CGGTTTTCTGGCAGCAGATCCGGCTCTGATTACAGCCTG |
| | ACCATCTCCTCTCTGGAGTCCGAGGACTTCGCCGATTAC |
| | TACTGCCTCCAGTATGCCAGCTACCCATATACATTTGGC |
| | GGCGGCACCAAGCTGGAGATCAAGTCTGACCCCGGCAG |
| | CGGCGGCGGCGGCTCCTGCCCCTACAGCAACCCTTCCC |
| | TGTGCTCTGGCGGAGGAGGCTCTTGTCCATATAGCAAT |
| | CCCAGCCTGTGCAGCGGCGGCGGCGGCAGCACCACAAC |
| | CCCAGCACCCAGACCCCCTACACCTGCACCAACCATCG |
| | CCTCCCAGCCTCTGAGCCTGCGGCCCGAGGCATGTAGA |
| | CCCGCTGCTGGAGGAGCCGTGCATACTAGAGGACTGGA |
| | CTTTGCTTGCGACATCTATATCTGGGCACCACTGGCCGG |
| | AACATGTGGCGTGCTGCTGCTGTCACTGGTCATTACACT |
| | GTACTGTAAGCGAGGCCGGAAGAAACTGCTGTATATTT |
| | TCAAACAGCCCTTTATGAGACCTGTGCAGACTACCCAG |
| | GAGGAAGACGGCTGCAGCTGTAGGTTCCCCGAGGAAG |
| | AGGAAGGCGGGTGTGAGCTGAGGGTCAAGTTTAGCCGC |
| | TCCGCAGATGCCCCTGCTTACCAGCAGGGGCAGAATCA |
| | GCTGTATAACGAGCTGAATCTGGGACGGAGAGAGGAAT |
| | ACGACGTGCTGGATAAAAGGCGCGGGAGAGACCCCGA |
| | AATGGGAGGCAAGCCACGACGGAAAAACCCCCAGGAG |
| | GGCCTGTACAATGAACTGCAGAAGGACAAAATGGCAG |
| | AGGCCTATAGTGAAATCGGGATGAAGGGAGAGAGAAG |
| | GCGCGGCAAAGGGCACGATGGCCTGTACCAGGGGCTGT |
| | CTACTGCCACCAAGGACACCTATGATGCTCTGCATATG |
| | CAGGCACTGCCTCCAAGGTGA |
| | (SEQ ID NO: 22) |
| CAR M26-R2 | GCTCTGCCCGTCACCGCTCTGCTGCTGCCACTGGCCCTG |
| | CTGCTGCACGCCGCCAGACCCGAAGTCCAGCTGCAACA |
| | GTCAGGCCCCGAACTGGTCAAACCCGGAGCCTCAGTCA |
| | AAATGTCATGTAAAGCCTCAGGATATACCTTCACATCCT |
| | ACTTCATCCACTGGGTGAAGCAGAAGCCTGGCCAGGGC |
| | CTGGAGTGGATCGGCTTCATCAACCCTTACAATGACGG |
| | CTCTAAGTATAACGAGAAGTTTAAGGGCAAGGCCACAC |
| | TGACCAGCGATAAGAGCAGCAGCACCGCCTACATGGAG |
| | CTGAGCAGCCTGACCAGCGAGGACTCCGCCGTGTACTA |
| | TTGCACAAGGGACGATGGCTACTATGGCTACGCTATGG |
| | ACTATTGGGGCCAGGGCACATCCGTGACCGTGTCTAGC |
| | GGCGGAGGAGGCTCCGGCGGAGGAGGCTCTGGCGGAG |
| | GAGGCAGCGACATCCAGATGACCCAGAGCCCCTCCTCT |
| | CTGTCTGCCAGCCTGGGAGAGAGGGTGTCCCTGACATG |
| | TCGCGCCACCCAGGAGCTGTCTGGCTACCTGAGCTGGC |
| | TCCAGCAGAAGCCTGACGGCACAATCAAGAGACTGATC |
| | TATGCCGCCTCCACCCTGGATTCTGGCGTGCCAAAGCG |
| | GTTTAGCGGCAATAGATCCGGCTCTGATTACTCCCTGAC |
| | CATCAGCTCCCTGGAGTCTGAGGACTTCGCCGATTACTA |
| | CTGCCTCCAGTATGCCATCTACCCATATACATTTGGCGG |
| | CGGCACCAAGCTGGAGATCAAGAGCGACCCCGGCTCCG |
| | GCGGCGGCGGCTCCTGCCCCTACTCTAACCCTAGCCTGT |
| | GCTCCGGCGGCGGGGGCTCTTGTCCATATTCTAATCCCA |
| | GCCTGTGCAGCGGCGGAGGAGGCAGCACCACAACCCC |
| | AGCACCCAGACCCCCTACACCTGCACCAACCATCGCCT |
| | CTCAGCCTCTGAGCCTGCGGCCCGAGGCATGTAGACCC |
| | GCTGCTGGCGGAGCTGTGCATACTAGAGGACTGGACTT |
| | TGCTTGCGACATCTATATCTGGGCACCACTGGCCGGAA |
| | CATGTGGCGTGCTGCTGCTGTCACTGGTCATTACACTGT |
| | ACTGTAAGCGAGGCCGGAAGAAACTGCTGTATATTTTC |
| | AAACAGCCCTTTATGAGACCTGTGCAGACTACCCAGGA |
| | GGAAGACGGCTGCAGCTGTAGGTTCCCCGAGGAAGAG |
| | GAAGGCGGGTGTGAGCTGAGGGTCAAGTTTAGCCGCTC |
| | CGCAGATGCCCCTGCTTACCAGCAGGGGCAGAATCAGC |
| | TGTATAACGAGCTGAATCTGGGACGGAGAGAGGAATAC |
| | GACGTGCTGGATAAAAGGCGCGGGAGAGACCCCGAAA |
| | TGGGAGGCAAGCCACGACGGAAAAACCCCCAGGAGGG |
| | CCTGTACAATGAACTGCAGAAGGACAAAATGGCAGAG |
| | GCCTATAGTGAAATCGGGATGAAGGGAGAGAGAAGGC |
| | GCGGCAAAGGGCACGATGGCCTGTACCAGGGGCTGTCT |
| | ACTGCCACCAAGGACACCTATGATGCTCTGCATATGCA |
| | GGCACTGCCTCCAAGGTGA |
| | (SEQ ID NO: 23) |

### 2.2 Assessment of CLL-1 Expression and Section of Target Cells

CLL-1 expression was checked on different tumor cell lines. HL-60 and U937 expressed the highest levels of CLL-1 amongst the tested cell lines. MOLM-13 and EOL showed an intermediate expression of CLL-1. We were not able to detect any antigen expression on JEKO, SupT1 and Jurkat cell lines. We further explored the non-specific lysis of these negative cell lines using non-transduced cells from two different donors.

Short and long term co-cultures of these cells with NT cells were performed for 4 h and 24h, respectively. At the end of this assay, it was pointed that JEKO generated lower background of cell lysis with NT than other cell lines. Thus, JEKO was selected as negative cell line for further tests and we kept HL-60 and U937 as positive cell lines and EOL as the cell line harboring intermediate expression.

CLL-1 expression on the different populations of PBMCs were investigated (Hemacare vials). CLL-1 was found to be highly expressed on monocytes in similar manner as CD33 and CD123. To the contrary, CLL-1 was not detected on T cells or B cells. The frequency of CLL-1 expressing cells (monocytes) decrease over time. It was not possible to identify any population expressing CLL-1 at 3 days post activation.

### 2.3 Assessment of CAR Expression and Activity

Primary T cells were activated and transduced with lentivectors produced in house (pSew-BFP). The CAR expression was assessed using different tools and at different timepoints. Although BFP was observed in different conditions, only four CARs were detected by other staining markers, i.e. Rituximab (RTX) and L-protein. BFP expression validated our lentivector productions and our transductions. While Fab didn't reveal the expression of any CAR, four different CARs were detected with L-protein and RTX. The staining with these latter showed a good correlation with BFP for the four different CARs. Therefore, these three methods were useful to monitor CAR expression. As B cells express CD20, and can be recognized by RTX, we have decided to assess CAR in further experiments as the double positive population for BFP and RTX. M5 showed the highest transduction efficiency while other candidates showed low transduction efficiency. However, the percentage of CAR+ increases over time for all candidates except 1075.7 by day 10 post-transduction. M2 yielded the highest percentage of CAR+ at the end.

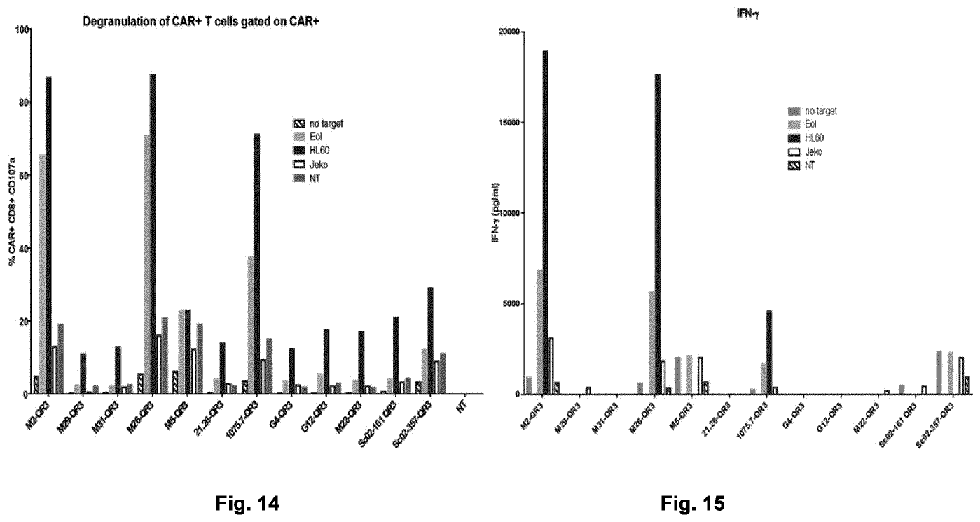

The specific activity of these CARs was evaluated with degranulation and cytotoxicity assays. After 6 hours of co-culture of CART cells with different target cells, M2 and M26 showed specific degranulation (above 30%) in response to antigen stimulation (FIG. 14). These CARs expressed high levels of CD107a. The degranulation activity of these two CARs was associated with IFN-α release (FIG. 15). Indeed, M2 and M26 released the highest amount of IFN- a in response to antigen stimulation with HL-60. Surprisingly, Sc02-161 and Sc02-357 produced IFN-α after co-culture with EOL cell line but not with the other target cells. Although Sc02-161 and Sc02-357 did not show any expression of CD107a in degranulation assay, EOL cells did induce the IFN- a production with Sc02-357 to higher level than other candidates.

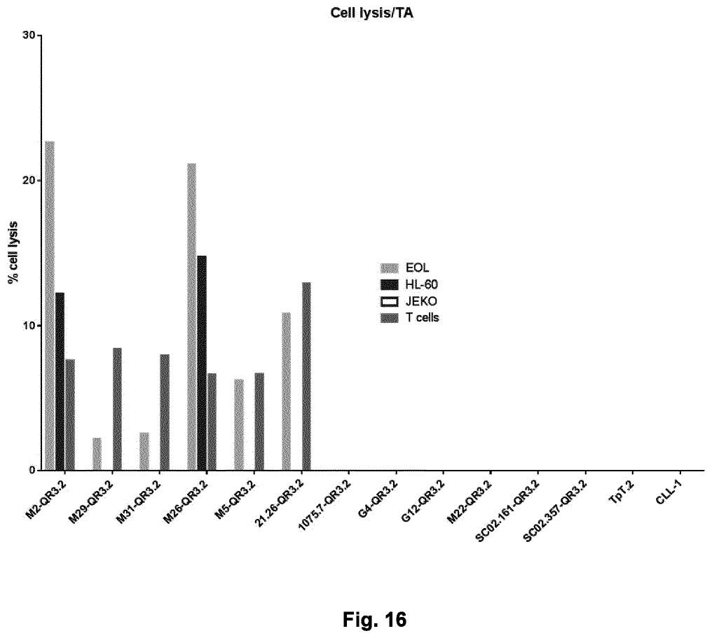

The cytotoxicity assay indicated that M2 and M26 were the only CARs exhibiting significant specific cell lysis with different target cell lines (FIG. 16). The level of cell lysis of HL-60, the most positive cell line, is around 25-30%. Non-transduced T cells were also used as target cells and no cell lysis was observed with different CAR T cells, suggesting that the level of CLL-1 expression on T cells is very low, if it is expressed.

Taken together, these data pointed out M2 and M26 as best candidates to move forward. The same analyzes were also performed with a second donor, confirming what was already observed with the first one. Also, M2 and M26 exhibited the highest enrichment of CAR+ T cells over time. Significant and substantial increase was observed between D11 and D14 post-transduction for M2 and M26 while other CARs showed slight increase over time. Although M5 exhibited the highest transduction efficiency, it demonstrated lower enrichment of CAR+ T cells than M2 and M26. These hallmarks were also observed with other donors.

The degranulation assay indicated that high level of CD107a was upregulated on the cell surface of several CAR T cells (M2, M26 and 1075.7) in response to antigen stimulation. The level of degranulation activity is higher than that observed with first donor, since the data presented here is gated on BFP+ cells. In addition, we didn't notice any degranulation background of T cells alone. The rest of CARs didn't show any degranulation signal.

Moreover, M2 and M26 were the only candidates showing specific significant IFN-γ secretion. Albeit 1075.7 expressed high levels of CD107a, its IFN-γ secretion was very low with different target cells. M2 and M26 CARs exhibited similar profile in both assays (degranulation and IFN-γ) and their response varied between different cell lines expressing different levels of antigen. In conclusion, M2 and M26 were the only candidates revealing significant degranulation activity associated to specific IFN-γ release. The difference of degranulation and IFN-γ level may be assigned to the difference of CAR+ T cells percentage at the end.

The cytotoxicity assay pointed out that M2 and M26 were the only candidates able to kill target cells with significant level of cell lysis above the threshold of 20%. Other candidates showed low levels of cytolytic activity (below the threshold). Interestingly, we observed low level of cell lysis of T cells (5%), suggesting that a low frequency of T cells may express CLL-1.

The previous experiments indicated that M2 and M26 are the candidates that met the required specification in term of specific activity. Moreover, these CARs showed an increase of the frequency of CAR+ T cells between D11 and D14 post-transduction. They had similar profile with different assays. Thus, with this third donor, we intended to characterize and discriminate these two CARs. In addition to previous assays (CAR expression, degranulation and cytotoxicity at day 10 post-transduction), we assessed the cytolytic activity of these CAR at different ratios and different timepoints. We have also added U937 as a positive target cell line.

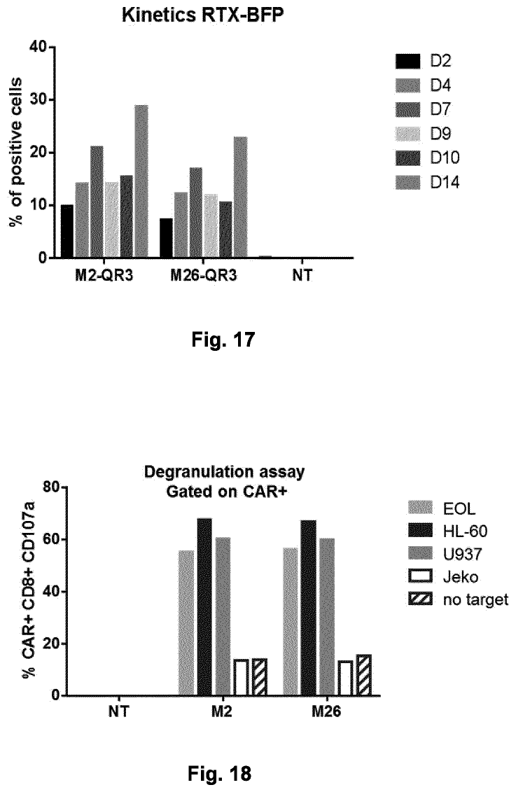

As already reported with previous donors, these CARs showed low transduction efficiency (around 10%). However, the percentage of CAR+ T cells increased over time and reached around 30% at the end for both candidates (FIG. 17). They demonstrated high degranulation activity with different cell lines (FIG. 18).

These CARs expressed similar levels of CD107a on CAR+ CD8+ T cells regardless the cell lines. The degranulation activity was not correlated with antigen density on different target cells.

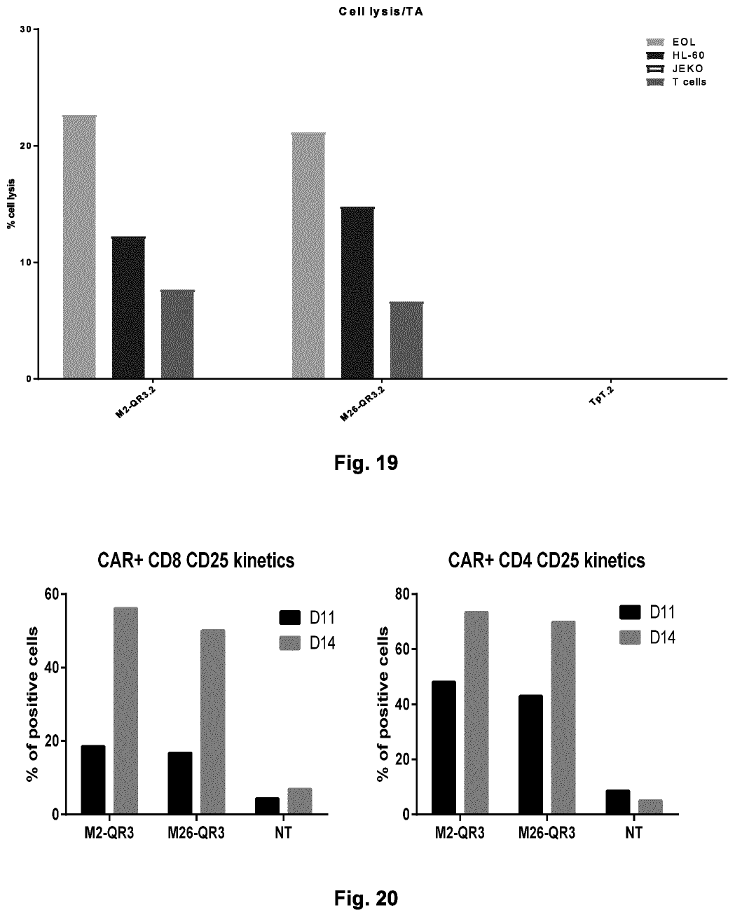

Cytolytic activity of CART cells at these two timepoints D10 and D14 was further explored, (FIG. 19). These candidates induced specific cell lysis and showed similar cytolytic activity at different ratios with several cell lines (HL-60 and U937). Although these CARs showed low levels of cell lysis with EOL cell line (around the threshold of 20%), M26 exhibited slightly higher cytolytic activity than M2 with this target cell (expressing the lowest level of antigen) at day 10 post-transduction. This difference was confirmed and emphasized at day 14 post-transduction.

We have also checked the expression of several markers in order to decipher the activation status and profile of these CARs between these two timepoints (D11 and D14). CAR T cells (BFP+) upregulated the activation marker CD25 on CD8 and CD4 CAR T cells between D11 and D14 (FIG. 20). While 30% of CART cells expressed CD25 at days 11, 70% and 55% of CD8 and CD4 T cells expressed CD25, respectively. However, both CARs showed similar levels of CD25 at different timings.

Although some discrepancies were observed between both candidates, they were not so different. They had similar profile in terms of degranulation and cytolytic activity although M26 had slightly higher cytolytic activity. All the experiments above were performed for CAR cloned in pSew-EF1α backbone.

Taken all together, these candidates are very similar. They had similar functional and phenotypic properties. The only potential difference is that M26 was less activated at day 14 post transduction. We noticed that the kinetics of CAR expression over time between D11 and D14 with this donor was different than that observed with previous donors.

### 2.4 Pooling Proliferation Assay

Since no obvious differences between the two different candidates M2 and M26 could be observed, it was decided to compare their antigen-dependent proliferation as per the pooling proliferation test of the present invention and see whether they would be correlated.

The M2 and M26 ScFv have been assayed under two different “R2” and “QR3” CAR architectures, which are depicted in FIG. 13, resulting into four CAR candidates:
anti-CLL1 CAR M2-QR3 (SEQ ID NO.20).
anti-CLL1 CAR M26-QR3 (SEQ ID NO.21).
anti-CLL1 CAR M2-R2 (SEQ ID NO.22).
anti-CLL1 CAR M26-R2 (SEQ ID NO.23).

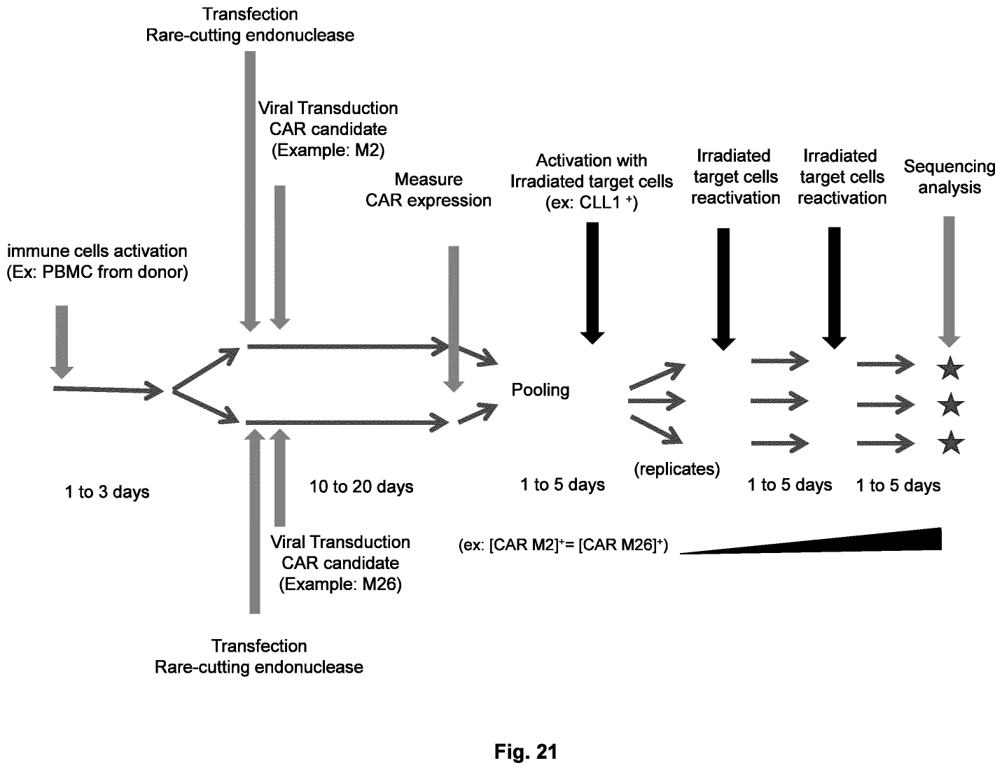

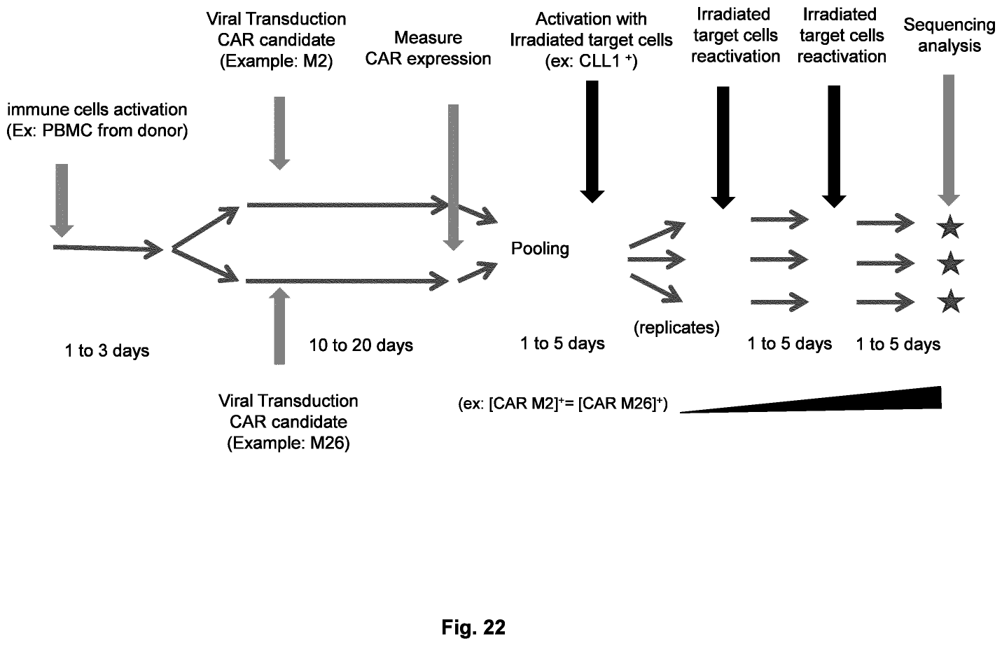

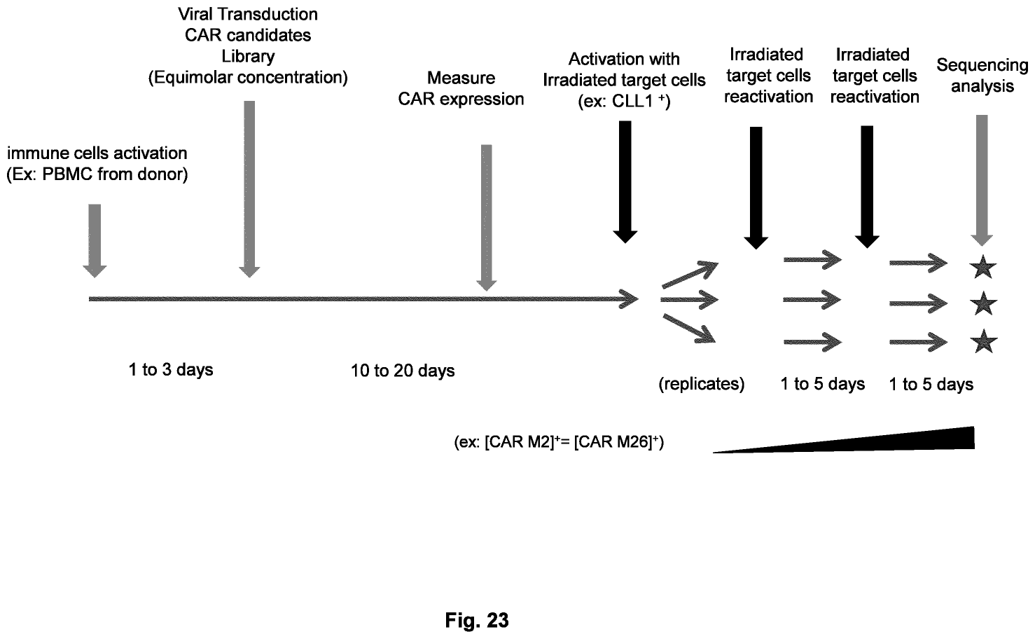

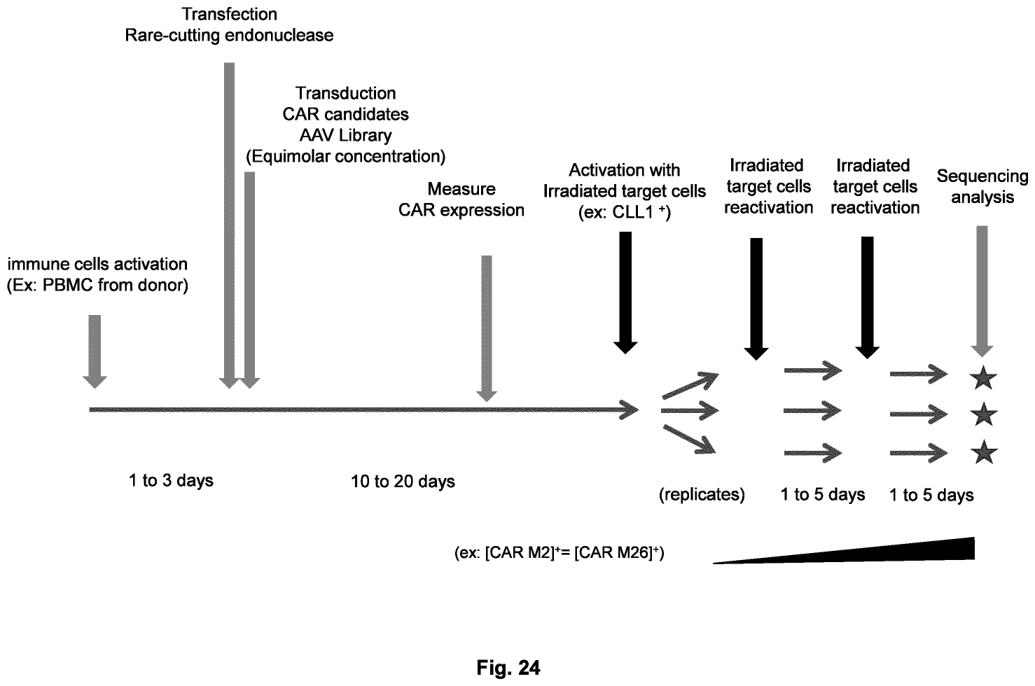

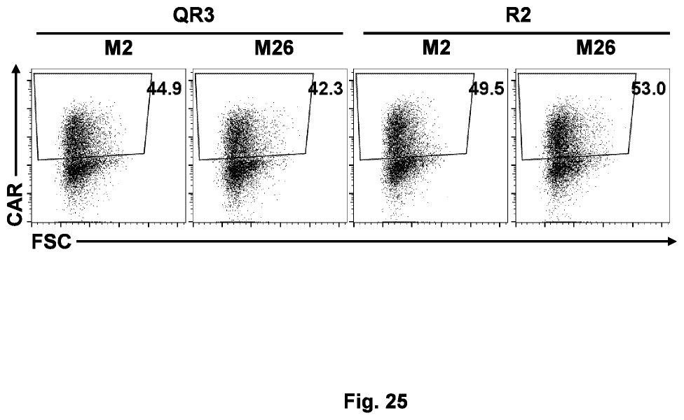

FIG. 21 summarizes the steps and time lines of this assay, which details are provided below.

Table 3 lists the polynucleotide sequences used in this example

PBMCs were thawed at day 0 and activated at day 1 using MACS GMP T Cell TransAct (0.06 mL of beads per 1×10⁶ CD3+ viable cells). Cells are cultured in X-Vivo 5% human serum IL-2 350 UI/mL. 3 days after activation, cells were split into different culture batches, each being transfected with 1 μg of total mRNA encoding TALE-nucleases targeting TCRalpha locus per 1×10⁶ cells as already described by Poirot et al. Cells were then seeded for 15 min at 37° C. before being transferred at 30° C. 1.5 h after electroporation, rAAV6 particles were added directly to the culture accordingly to the MOI chosen. The four cultures were respectively transduced with the following rAAV6 particles:
rAAV6 anti-CLL1 CAR M2-QR3 comprising SEQ ID NO.20 flanked by polynucleotide sequences homologous to TCRalpha genomic region.
rAAV6 anti-CLL1 CAR M26-QR3 comprising SEQ ID NO.21 flanked by polynucleotide sequences homologous to TCRalpha genomic region.
rAAV6 anti-CLL1 CAR M2-R2 comprising SEQ ID NO.22 flanked by polynucleotide sequences homologous to TCRalpha genomic region.
rAAV6 anti-CLL1 CAR M26-R2 comprising SEQ ID NO.23 flanked by polynucleotide sequences homologous to TCRalpha genomic region.

Cells were cultured overnight at 30° C. and then transferred back at 37° C. before washing the virus with fresh medium. Cells were then cultured for 15 days and concentration of CAR positive cells were assessed by flow cytometry in each batch as shown in FIG. 25.

In parallel, positive (HL60, CLL1+) and negative control (Jekol, CLL1-) target cells were irradiated in T25 flask at 60Gy using Compact X-Ray Irradiation System-CellRad, Faxitron #2328A50149. Cells were then centrifuged at 1500 rpm for 5 mn and resuspended at 0.5×10⁶ cells/mL in X-VIVO™ 15, FBS 10%, IL-2 35 UI/mL culture media.

At the end of the initial subcultures, a pool of the four different CAR candidates cells which had close frequencies of CAR expression, were centrifuged for 5 min at 300 g and resuspended in 1.5 mL of X-Vivo-15 media supplemented by 35UI/ml IL-2 and 10% FBS. Cell densities were then determined using LUNA™ Automated Cell Counter/Trypan method and mixed together using an equimolar ratio at 0.5×10⁶ CAR+cells/mL in X-VIVO™ 15, FBS 10%, IL-2 35UI/mL culture media.

The pooled culture of CAR positive cells was then cocultured with irradiated HL60 or Jekol target cells (0.5×10⁶ cells from an equimolar mix of CAR CLL1 positive cells+0.5×10⁶ target cells) in 2 mL of X-VIVO™ 15, FBS 10%, Il-2 35 UI/mL culture media in 24 well-plates (each condition was performed in triplicates). The same mix of cells, HL60 and Jekol cells cultured alone were used as controls (one well per condition). This first coculture corresponded to a new day 0 of this serial CAR selection experiment. At this time point, 1×10⁶ cells from each group of CAR CLL1 positive cells were harvested and cell pelleted, 3×10⁶ of irradiated HL60 or irradiated Jekol cells were harvested and cell pelleted, as well as 3x10⁶ of mixed CAR CLL1 positive cells.

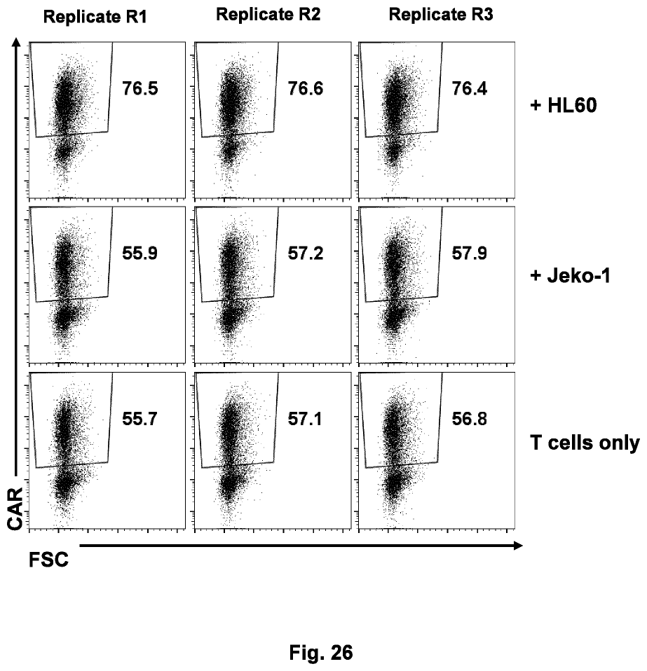

4 days later, cell densities were determined using LUNA™ Automated Cell Counter/Trypan method in each well of the coculture. At day 4, more than 75% of the mix of the cells were CAR+ after reactivation by positive (HL60) target cells (FIG. 26). When cells were cocultured with negative (Jekol) target cells, the frequency of CAR+ cells slightly increased but did not reach the level reached with HL60 cells. One more reactivation step was then performed at the same E:T 1:1 ratio as previously described. The mix of CAR-T cells was cocultured with irradiated HL60 or Jekol target cells (0.5×10⁶ cells of CAR-T cells+0.5×10⁶ target cells) in 2 mL of X-VIVO™ 15, FBS 10%, Il-2 35 UI/mL culture media in 24 well-plates. Before reactivation, 1×10⁶ cells from each group of cells cocultured either with positive or negative target cells were harvested and cell pelleted. At this time point (d4), 1×10⁶ of T cells alone (mix) were also harvested and cell pelleted as control.

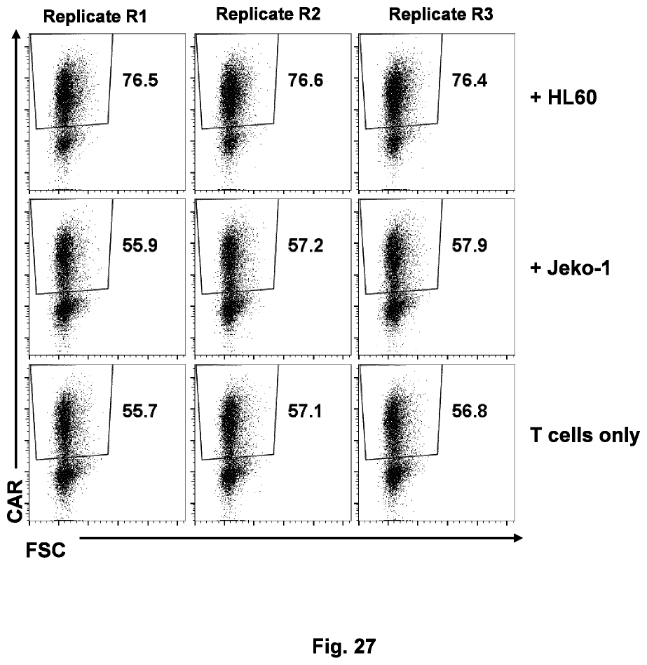

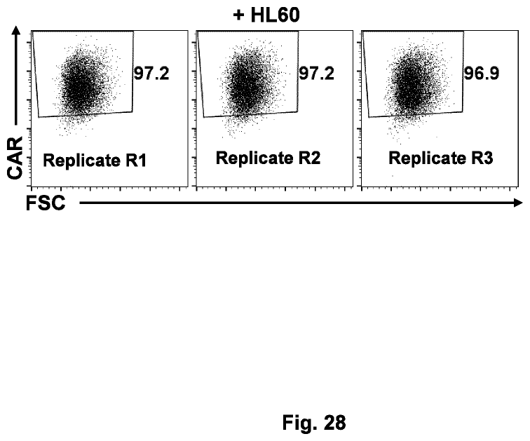

3 days later (day 7), cell densities were determined using LUNA™ Automated Cell Counter/Trypan method in each well of the coculture. More than 90% of the UCARTGT CLL1 cells were CAR+ after reactivation by positive (HL60) target cells (FIG. 27).

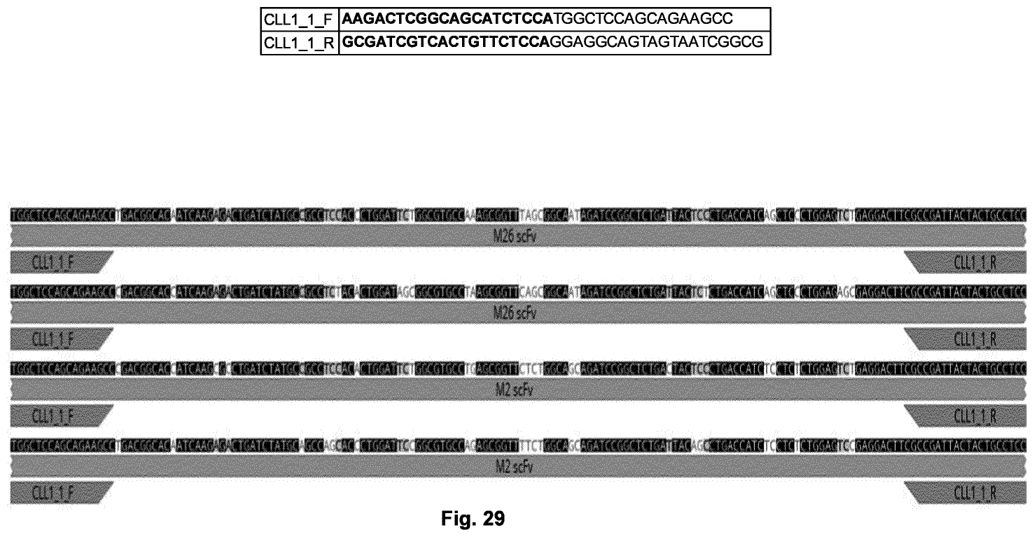

The following day (day 8), anti-CLL1 CAR T-cells were then reactivated at the same E:T 1:1 ratio as previously described. 3 days later (day 11), cell densities were determined using LUNA™ Automated Cell Counter/Trypan method in each well of the coculture. At day 11, 3 days after the third reactivation, more than 96% of the mix of anti-CLL1 CAR T-cells was CAR+ after reactivation by positive (HL60) target cells (FIG. 28).

At day 11, 1×10⁶ cells from each group of CAR positive cells co-cultured either with positive target cells were harvested and cell pelleted.

In this whole process, cell pellets were harvested at day 0, day 4, day 8 and day 11. gDNA was extracted from all the samples using the DNeasy Blood & Tissue Kit following the manufacturer's instructions.

A set of primers, which sequences are shown in Table 3 were designed so as to be able to discriminate the different CARs by deep sequencing analysis.

The anti-CLL1 studied CAR candidates show many differences that would be detectable by deep sequencing. However since only few regions are conserved between the scFv of M2 and M26, a set of primers were designed in which the forward and reverse primers are located in small conserved regions of the scFv (FIG. 29). Then, PCR amplification was performed for each sample. The level of amplification was then assessed by migration on agarose gel. Positive samples, as defined by the presence of a band on 1% agarose gel, were purified using AMPure. As expected, no amplification was detected for gDNA samples corresponding to HL60 or Jeko 1 irradiated cells alone or to non-transduced T cells. DNA concentration was determined using the Quant-iT™ PicoGreen® dsDNA Assay Kit and samples were sent for Illumina sequencing to an external platform at ICM.

Sequencing by Illumina MiSeq gave an average of 141,291 analyzable reads (standard deviation of 25,625). These reads were aligned against the sequences of the different scFv and a score of alignment was calculated. The results demonstrate that there is no dramatic enrichment of any CAR above another overtime when the pool is cocultured in the presence of positive target cells, which correlates with the previous results obtained by the classical analysis performed in Example 2.3.

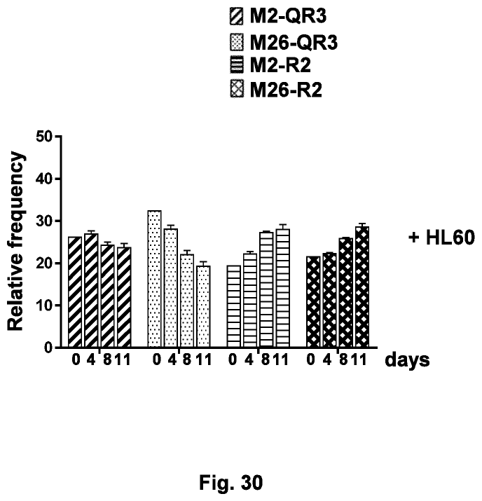

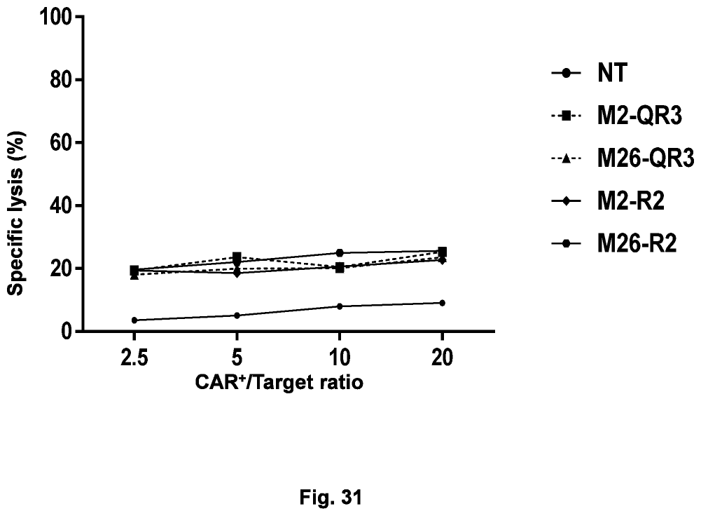

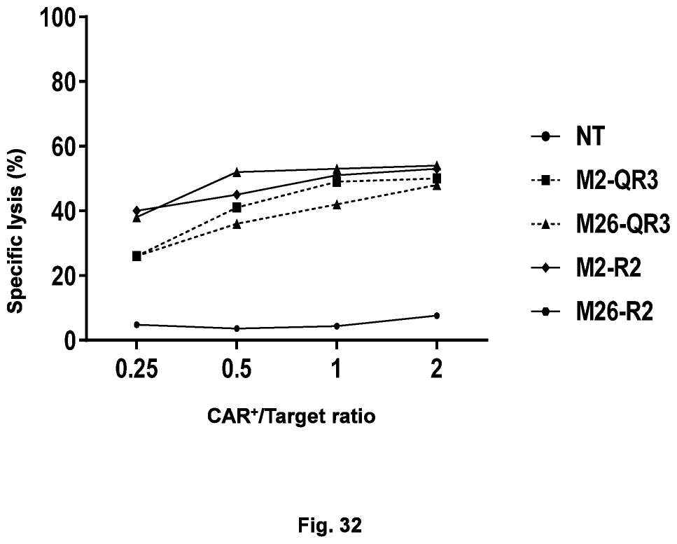

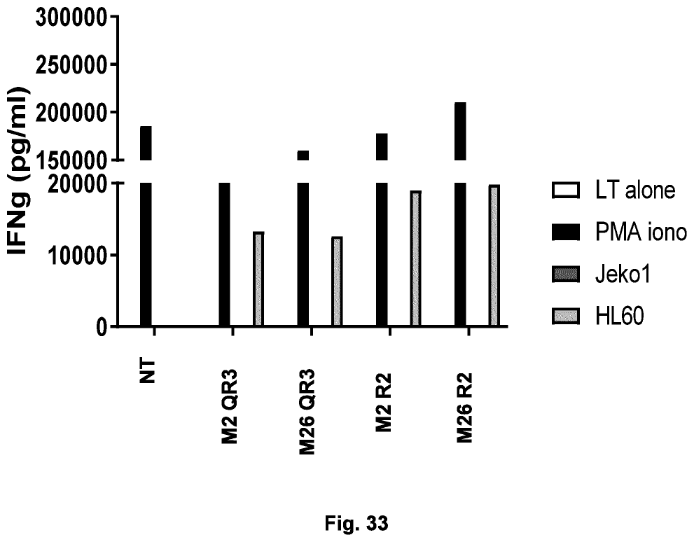

Although M2 and M26 provide comparable CAR potencies under both architectures, the CAR architecture R2 was found as per the present method to confer relative advantage over the architecture QR3. This could be more particularly observed from the results shown in FIGS. 30, 32, and 33.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1-92. (canceled)

93. An in vitro method to compare antigen dependent activation/proliferation of allogeneic CAR immune cells sub-populations that have different genetic background, said method comprising:
i) providing sub-populations of immune cells endowed with a at least one CAR, wherein said CAR of each subpopulation targets the same antigen;
ii) incubating the sub-populations of CAR immune cells under i) with target cells expressing said antigen for a period of time;
iii) adding an additional quantity of target cells to the incubated CAR immune cells and incubating for an additional period of time;
iv) detecting the presence of an enriched sub-population(s) of CAR immune cells encoding said at least one CAR; and
v) selecting said enriched sub-population(s) of CAR immune cells.

94. The method of claim 93, wherein said sub-populations of T-cells are provided from different donors.

95. The method of claim 93, wherein said method comprises an initial step of activating the sub-population of immune cells with one or more T-cell stimulating agents.

96. The method of claim 93, wherein said target cells are replication deficient.

97. The method of claim 96, wherein said target cells are irradiated cells.

98. The method of claim 93, wherein said enriched sub-population is detected by sequencing or amplifying the polynucleotides encoding said CAR(s).

99. The method of claim 93, wherein the quantity of CAR immune cells and/or target cells is determined by assaying for the presence of a detectable label.

100. The method of claim 93, wherein the quantity of CAR immune cells and/or target cells is determined by flow cytometry and/or cell counting.

101. The method of claim 93, wherein the concentration of interferon gamma is assayed during and/or after the incubation steps ii) and iii).

102. The method of claim 93, wherein the period of time of steps ii) and iii) ranges from about 12 hours to about 120 hours.

103. The method of claim 93, wherein step iii) is repeated from 1 to 50 times.

104. The method of claim 93, wherein the CAR immune cells are incubated with the target cells at a ratio of about 1:1 to about 1:16.

105. The method of claim 93, wherein the enriched sub-population of CAR immune cells is detected by PCR using a primer set that is specific for the enriched CAR immune cell sub-population.

106. The method of claim 93, wherein the antigen is selected from the group consisting of CD19, CD22, CD123, and CS1.

107. The method of claim 93, wherein the CAR immune cells are resistant to one or more chemotherapeutic agents.

108. The method of claim 93, wherein the CAR immune cells comprise an inactivating mutation in their genes encoding TCRalpha and/or TCRbeta to make them allogeneic.

109. The method of claim 93, wherein the CAR immune cells comprise an inactivating mutation in CD52 to make them resistant to Alemtuzumab.

110. The method of claim 93, comprising repeating step iii) one or more times.
